(19) United States
(12) Patent Application Publication
(10) Pub. No.: US 2025/0363814 A1
(43) Pub. Date: Nov. 27, 2025

(54) Fatigue Discovery Analysis

(71) Applicant: Blueskeye AI Ltd, Nottingham (GB)

(72) Inventors: Michel François Valstar, Nottingham (GB); Anthony Brown, Nottingham (GB); Thomas James Smith, Sheffield (GB); Tze Ee Yong, Nottingham (GB); Mani Kumar Tellamekala, Nottingham (GB); Shashank Jaiswal, Nottingham (GB); Adrian Marinescu, Nottingham (GB)

(21) Appl. No.: 19/213,885

(22) Filed: May 20, 2025

Related U.S. Application Data
(60) Provisional application No. 63/651,390, filed on May 23, 2024.
(60) Provisional application No. 63/721,963, filed on Nov. 18, 2024.

(51) Int. Cl. G06V 20/59

(52) U.S. Cl. CPC G06V 20/597

(57) ABSTRACT

Described is a system to evaluate the efficacy of a set of candidate interactive tasks in eliciting fatigue markers in facial and vocal expressions. Input video and audio streams are processed, followed by face detection, followed by detecting the facial landmarks. Based on these, higher level features are derived, such as gaze vectors, head pose, action units and audio features based on the audio input. Higher level features are fused together over a time window and used to estimate the fatigue level as well as a confidence level for the estimate. The video and audio streams may either be collected organically (e.g., in a car/plane) or collected with the help of an app that presents the user with a stimulus task.

### PRIOR APPLICATIONS

This application claims the benefit of the following two applications, which are incorporated by reference in their entirety:
1. U.S. Provisional Patent Application No. 63/651,390, filed on May 23, 2024; and
2. U.S. Provisional Patent Application No. 63/721,963, filed on Nov. 18, 2024.

### FIELD OF THE DISCLOSURE

The present disclosure relates generally to improved techniques to improve the detection of fatigue based on facial and vocal expressions.

### BACKGROUND

People are generally good at self-assessing their fatigue level by using various self-reporting tools. In some cases, these approaches could interfere with the primary task (e.g., driving, flying) and so, having a method that can provide this information in real time and without interrupting the task could provide great benefits. In the automotive and aerospace domain, having individual level longitudinal fatigue data could allow us to better understand how various factors (e.g., shift patterns, driving automation level) might impact fatigue levels for each individual. This would allow making more informed decisions when designing such systems, as well as, at a more granular level, providing feedback to automation systems (such as in the case of autonomous vehicles).

Another need is for evaluating the impact of various medical products during clinical trials on patients.

Fatigue estimation would be beneficial also for improving depression diagnoses.

### SUMMARY

The objectives of this disclosure include:
To evaluate the efficacy of a set of candidate interactive tasks in eliciting fatigue markers in facial and vocal expressions;
To recommend a subset of the evaluated tasks for incorporation into the Mobile App to be used in Fatigue data collection.

Our system, presented below, takes input video and audio streams, it performs face detection, followed by detecting the facial landmarks. Based on these, higher level features are derived, such as gaze vectors, head pose, action units and audio features based on the audio input. Higher level features are fused together over a time window and used to estimate the fatigue level as well as a confidence level for the estimate.

The video and audio streams can either be collected in the wild (e.g., in a car/plane) or collected with the help of an app that presents the user with a stimulus task.

Analysis from data lake entries pertaining to facial and vocal expressions may be representative of apparent fatigue from a Discovery Phase. Also included are directions for the interactive tasks to be incorporated in a Mobile App, expressive behavioral markers of fatigue extracted from face and voice data, software development kit, or web-accessible platform for the detection of fatigue.

### BRIEF DESCRIPTION OF THE DRAWINGS

The accompanying figures, where like reference numerals refer to identical or functionally similar elements throughout the separate views, together with the detailed description below, are incorporated in and form part of the specification, serve to further illustrate embodiments of concepts that include the claimed invention and explain various principles and advantages of those embodiments.

FIG. 1 is flowchart of a model to estimate fatigue levels.

FIG. 2 is a schematic of a phone held in the user's hand.

FIG. 3 is a schematic of a phone screenshot having a moving white circle.

FIG. 4 is a schematic of a phone screenshot having a moving reading passage.

FIG. 5 is a schematic of a phone screenshot having an image.

FIGS. 6A, 6B, 6C, and 6D are a spread of facial expressions.

FIG. 7 is a graph of time of day versus fatigue score.

FIG. 8 is a graph of mean reaction time versus fatigue score.

FIGS. 9A and 9B are a spread of a pictures of how to hold a phone.

FIG. 10 is a picture of how not to hold a phone.

FIG. 11 is a graph of time versus angle for saccades.

FIG. 12 is a schematic of a face overlay.

FIG. 13 is a graph of fatigue score versus saccade velocity.

FIG. 14 is a boxplot of saccade velocity.

FIG. 15 is a graph of time versus normalized power for blinks.

FIG. 16 is a boxplot of blink phase 3 duration.

FIG. 17 is a boxplot of blink phase 3 area under the curve.

FIG. 18 is a boxplot of blink phase 5 area under the curve.

FIG. 19 is a boxplot of audio loudness.

FIG. 20 is a boxplot of audio mean pause counts.

FIG. 21 is a graph of time versus angle for saccades.

FIG. 22 is a boxplot of saccade velocity.

FIG. 23 is a boxplot of blink rate.

FIG. 24 is a boxplot of blink duration.

FIG. 25 is a boxplot of closure phase duration.

FIG. 26 is a spread of action units of the lower half of the face.

FIG. 27 is a boxplot of AU 6 (Cheek Raiser).

FIG. 28 is a boxplot of AU 10 (Upper Lip Raiser).

FIG. 29 is a boxplot of AU 12 (Lip Corner Puller).

FIG. 30 is a boxplot of AU 14 (Dimpler).

FIG. 31 is a boxplot of AU 15 (Lip Corner Depressor).

FIG. 32 is a boxplot of AU 17 (Chin Raiser).

FIG. 33 is a boxplot of AU 23 (Lip Tightener).

FIG. 34 is a boxplot of valence.

FIG. 35 is a boxplot of audio loudness.

FIG. 36 is a boxplot of audio mean pause counts.

FIG. 37 is a boxplot of blink duration.

FIG. 38 is a boxplot of blink phase 5 duration.

FIG. 39 is a boxplot of opening phase duration.

FIG. 40 is a boxplot of blink rate

FIG. 41 is a boxplot of AU 6 (Cheek Raiser).

FIG. 42 is a boxplot of AU 10 (Upper Lip Raiser).

FIG. 43 is a boxplot of AU 12 (Lip Corner Puller).

FIG. 44 is a boxplot of AU 14 (Dimpler).

FIG. 45 is a boxplot of AU 15 (Lip Corner Depressor).

FIG. 46 is a boxplot of AU 17 (Chin Raiser).

FIG. 47 is a boxplot of AU 23 (Lip Tightener).

FIG. 48 is a task flow time diagram.

FIG. 49 is distribution plot of fatigue levels.

FIG. 50 is a correlation plot of fatigue labels.

FIG. 51 is a boxplot of fatigue model precision.

FIG. 52 is a distribution of reaction time by condition.

FIG. 53 is a distribution of reaction time by condition and time.

FIG. 54 is a distribution of reaction time by condition and time.

FIG. 55 is a boxplot of blink closure features.

FIG. 56 is a boxplot of saccade speed.

FIG. 57 is a correlation plot of gaze features and reaction time.

FIG. 58 is a boxplot of arousal.

FIG. 59 is a boxplot of dominance.

FIG. 60 is a temporal graph of a drowsiness model score.

FIG. 61 is a progression of average drowsiness scores.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Relying on high level features such as gaze vectors, head posture, facial action units and audio features makes our model highly interpretable.

1. Using our approach, collecting longitudinal fatigue data without the need of self-reporting could be done in car while driving (or any other vehicle: train/plane) or using a phone app and:
be integrated by wellbeing apps and allow for a more in depth understanding of someone's experience;
be used in the context of autonomous or assisted driving to better understand the state of the driver;
be used in the context of informing a depression diagnosis; or
be used for diagnosing myalgic encephalomyelitis or chronic fatigue syndrome.

2. Collecting data in clinical studies to assess the effect of certain medication on fatigue levels.

3. Longitudinal data could offer more insight into fatigue patterns and allow for better system design (e.g.: shift patterns for safety critical domains: aerospace, medicine, nuclear).

I. INTERACTIVE TASKS FOR FATIGUE ANALYSIS

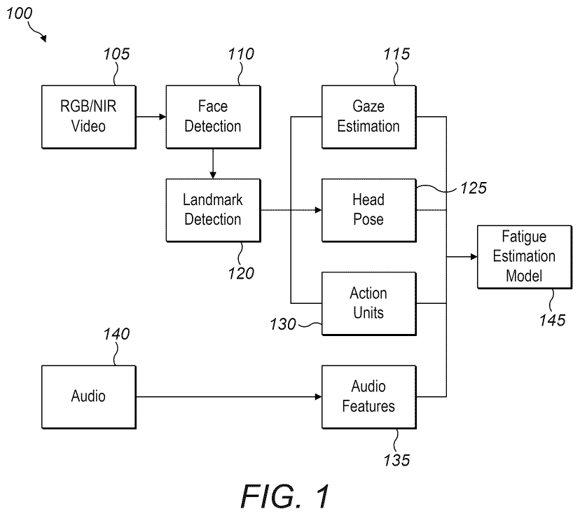

Turning to FIG. 1, shown is flowchart 100 of the model to estimate fatigue levels. The model can accept either RGB (red, green, blue) or NIR (near-infrared) video input to estimate fatigue levels. Input RGB or NIR video 105 is fed into a face detection module 110, which is fed into a landmark detection module 120. This is then fed into a gaze estimation module 115, a head pose module 125, and an action units module 130. Separately, audio 140 is fed into an audio features module 135. A fatigue estimation model 145 processes the output of the gaze estimation module 115, the head pose module 125, the action units module 130, and the audio features module 135.

Our approach relies on multiple facial features, derived from gaze patterns, head movement patterns, action unit activation intensity and speed as well as audio features. While facial and voice features have been used before, the most widespread facial features focus on eye aspect ratio, mouth aspect ratio, yawn frequency [1] [2] [3]. The main benefits of this approach are the use of gaze movement features (known to be sensitive to changes in fatigue [4]), action unit features combined with all the other previously used features.

A. Tasks Considered

We considered the following five tasks for evaluation and refinement:

Task 1. Reaction Time

This task intends to capture the user's mental fatigue by measuring their response time to a static stimulus. In the current implementation of this task, the user is asked to press a static visual stimulus displayed on a touch screen as soon as it appears on the screen. This task lasts for 30 to 120 seconds and it is performed with the phone held in the user's hand.

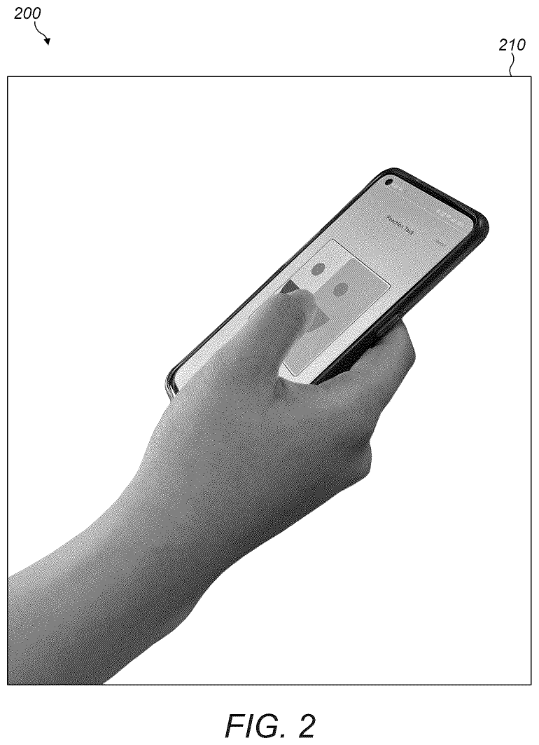

Turning to FIG. 2, shown is a schematic 200 of an illustration 210 of a phone held in the user's hand.

Task 2. Gaze Tracking

This task focuses on the user's eye gaze shift dynamics for estimating fatigue and cognitive load. In the current implementation of this task, the user is asked to move only their eye gaze to follow a moving circle on the screen.

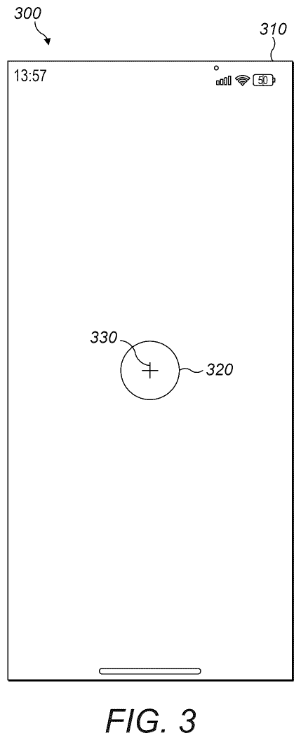

Turning to FIG. 3, shown is a schematic 300 of a phone screenshot 310 having a moving white circle 320 with a plus (“+”) sign 330 in the middle.

The gaze tracking task lasts for 25 to 60 seconds, using a tripod will increase the duration by the time it takes to set the phone on the stand from the previous hand held position during the reaction time task (this should not take more than a few seconds).

The image below shows a screenshot of the gaze tracking task, the white circle moves from top to bottom and once it reaches the bottom of the screen it disappears and appears at the top, this will induce a saccade movement as can be seen in the later graphs. The circle also has a plus (“+”) sign in the middle which helps participants more easily fix their gaze to the circle.

With the objective of estimating the user's cognitive load or mental fatigue, this task may include additional elements. The circle may have a number, shape, or symbol in it. The circle may be of different colors. The user may be asked to only follow those circles that satisfy a mathematical or logic rule, for example only following blue circles or circles that have a prime number on them. The user may be asked to indicate that the circle satisfies a rule in a different way, for example tapping the screen, or displaying a prototypical facial expression (e.g., a smile).

Task 3. Read Aloud

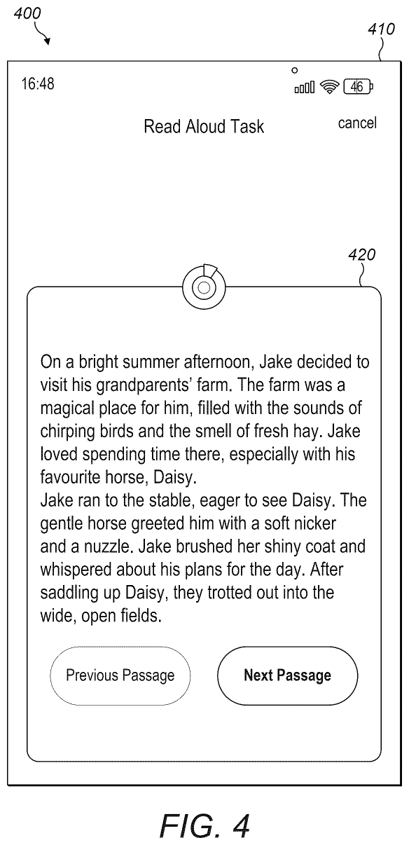

Turning to FIG. 4, shown is a schematic 400 of a phone screenshot 410 having a moving reading passage 420.

This task is intended to measure changes in vocal characteristics induced by fatigue. The current implementation of this task involves the user reading multiple pages of text, as shown below. To avoid the memorization of the shown text, it is ensured that the presented text is different each time a user conducts this task.

Task 4. Picture Description

This task focuses on analyzing changes in both facial and vocal muscle activity features caused by fatigue.

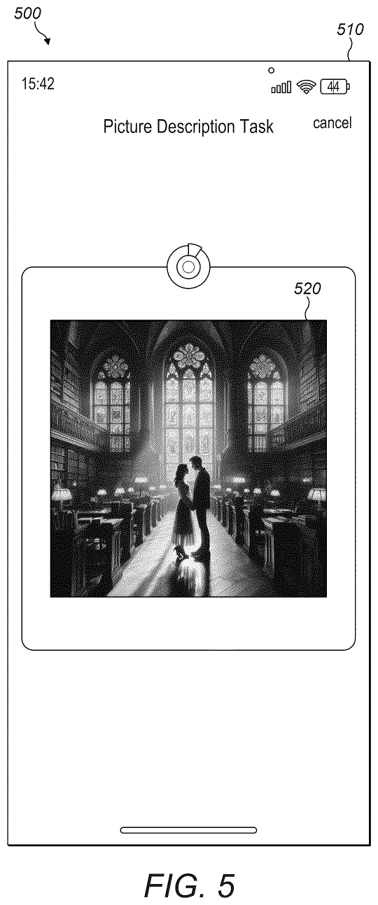

Turning to FIG. 5, shown is a schematic 500 of a phone screenshot 510 having an image 520.

In the current approach to this task the user is asked to describe a picture shown on a screen for 40 to 180 seconds. Similar to the Read Aloud task, the presented pictures are ensured to be different across the sessions.

Task 5. Expression Mimicry

This task aims to capture fatigue-induced changes in facial muscle dynamics through expression mimicking.

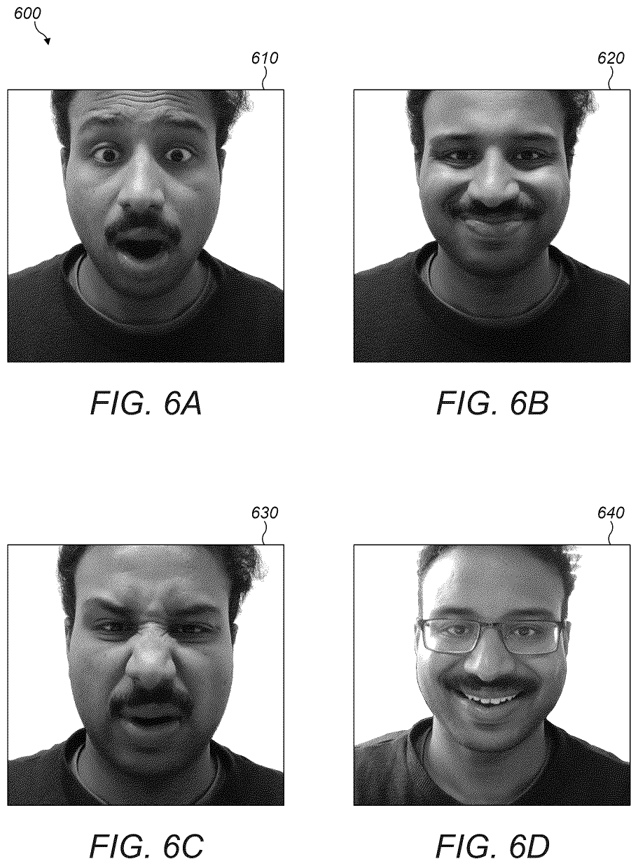

Turning to FIGS. 6A, 6B, 6C, and 6D, shown is a spread 600 of facial expressions 610 620 630 640 for the user to mimic.

In the current implementation of this task the user is asked to mimic the facial expressions that correspond to different facial action units and their intensity levels.

B. Fatigue Questionnaire

A general fatigue questionnaire is used as part of the Discovery Phase data collection as a point of reference for potential fatigue markers measured from the data collected using the candidate tasks.

The Chalder Fatigue Scale (CFS) [1], used as a reference in the Discovery Phase data collection, is a self-administered questionnaire for measuring fatigue levels in both clinical and non-clinical populations. It has 11 items that each have a 4-point scale: “less than usual”, “no more than usual”, “more than usual”, and “much more than usual”.

The 11-item version is the most used version today:

1. Do you have problems with tiredness?

2. Do you need to rest more?

3. Do you feel sleepy or drowsy?

4. Do you have problems starting things?

5. Do you lack energy?

6. Do you have less strength in your muscles?

7. Do you feel weak?

8. Do you have difficulties concentrating?

9. Do you make slips of the tongue when speaking?

10. Do you find it more difficult to find the right word?

11. How is your memory?

Before each data collection session, participants were first asked to fill in the questionnaire. The total score for this questionnaire can vary between 0 and 33, with 33 representing the highest level of fatigue.

II. TASK-WISE FATIGUE CANDIDATE MARKERS AND FEATURES

For the purpose of evaluating interactive tasks in terms of their ability to elicit fatigue cues in facial and vocal expressions, we first identified some relevant fatigue candidate markers and then derived their corresponding features for each task. A fatigue marker encompasses a broad range of facial or vocal characteristics indicating fatigue, while a fatigue feature is a specific, measurable parameter corresponding to a given marker.

TABLE 1 shows the list of interactive tasks and their corresponding fatigue markers and features used in Discovery Phase data analysis.

TABLE 1

| Interactive | | |
|---|---|---|
| Task | Fatigue Candidate Markers | Fatigue Candidate Features |
| Reaction | Time difference stimulus | Per-session average response |
| Task | presentation and user | time |
| | reaction | |
| Gaze | Saccades | Saccade angular velocity |
| Tracking | Blinks | Blink phase durations |
| Read Aloud | Speech parameters | Loudness variability and |
| | | Speech articulation rate |
| | Saccades | Saccade angular velocity |
| | Blinks | Blink rate and duration |
| Picture | Facial muscle activations | Maximum AU-wise intensity |
| Description | Reduced facial expression | Valence standard deviation |
| | intensity | |
| | Speech parameters | Loudness variability and |
| | | Speech articulation rate |
| | Blinks | Blink rate and duration |
| Expression | Facial muscle movements | Maximum AU-wise intensity |
| Mimicry | | |

As shown in Table 1 above, for each task considered for evaluation in the Discovery Phase we derived a set of fatigue markers and their features from the collected audiovisual recordings (except for the Reaction Task). Note that these are only some representative fatigue markers and features that are considered for task evaluation purposes, and the facial and vocal features that will be extracted from the POC phase dataset will include additional sets of features as specified in the sample datasheets.

Detailed descriptions of the computation of these fatigue features from AV data are presented in the results and analysis section.

To assess how well an audiovisual interactive task is able to elicit fatigue markers in face and voice data, we used the following criteria:
Is it possible to measure at least one fatigue feature from the audiovisual recordings captured using a given task?
   For example, in the Gaze Tracking task this translates to the feasibility of being able to extract saccadic and blink parameters from eye gaze directions and landmark coordinates estimated using an SDK.
If so, are the measured fatigue features of a given task qualitatively distinct between representative low and high fatigue classes?
   In the Gaze Tracking task, answering this question involves (a). Comparing the saccadic angular velocity, blink rate and duration features of low and high fatigue classes and (b). Qualitatively analyzing the feature differences between the two classes.

III. DATA PREPARATION FOR TASK EVALUATION

A. Fatigue Data Entries Selection Criteria for Task Evaluation

For task evaluation purposes, we used the existing fatigue data entries a data lake. Considering that this fatigue data was collected from the general population, it is expected to be highly noisy in terms of the self-reported fatigue scores. We evaluated the algorithms on the data collected from 20 sessions.

The comparisons that are presented in the report are between low and high fatigue levels. First of all, the design of the study made it more likely that we will sample low and high fatigue values, by explicitly asking participants to record data at any time of day that would ensure this for them. Furthermore, low fatigue levels were considered to be below rating 12, while high fatigue was considered over 16 on the Fatigue score scale.

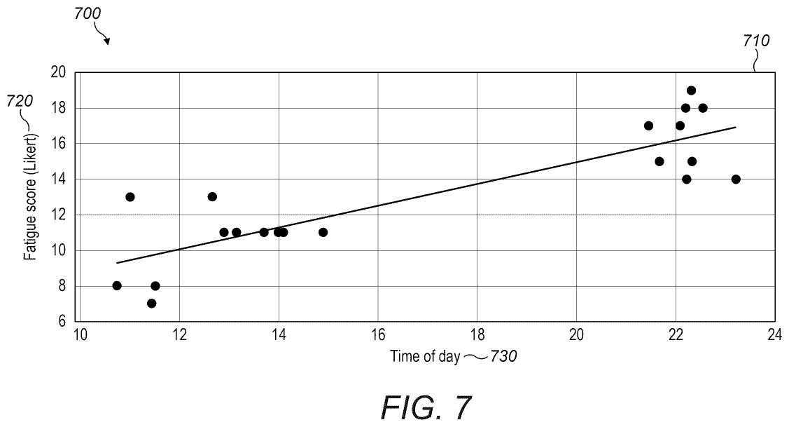

Turning to FIG. 7, shown is a graph 700 of a plot 710 having the time of day 730 as the x-axis and the fatigue score (using a Likert scale) 720 as the y-axis. The plot 710 shows, by design, the collected data was roughly clustered into low and high fatigue scores.

A Likert scale is a rating scale used to measure opinions, attitudes, or behaviors by presenting respondents with a range of answer options along a spectrum, typically ranging from “strongly disagree” to “strongly agree”. It is commonly used in surveys and questionnaires to gather qualitative data that can be quantified and analyzed.

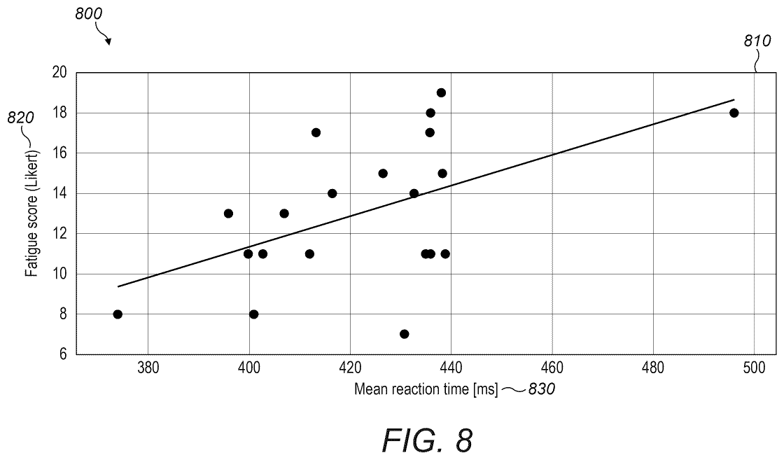

Turning to FIG. 8, shown is a graph 800 of a plot 810 having the mean reaction time in ms 830 as the x-axis and the fatigue score (using a Likert scale) 820 as the y-axis.

The graph 800 in FIG. 8 shows the same sample of fatigue scores as in the graph 700 in FIG. 7 but this time against the mean reaction time (obtained from the reaction time task). We observe a good correlation with the fatigue scores. This sample of reaction time also opens the question of how sensitive this measure is for higher fatigue levels (as assessed by the Chalder Fatigue Scale (CFS)), or whether the relationship between the two is linear over the entire interval.

B. Instructions to the Users During Fatigue Data Collection

To ensure that the collected data entries in the fatigue data lake have good quality audiovisual recordings, the following instructions were provided to the participants. Task 1. Reaction Task

Instructions may include the following.
Please press a static visual stimulus with the thumb of your dominant hand, which will be held hovering close to the screen of your phone.
The image below shows an example of how to hold your phone during this task (for a right handed person).
Camera Positioning. The aim of this task is to offer guidance about the phone positioning.

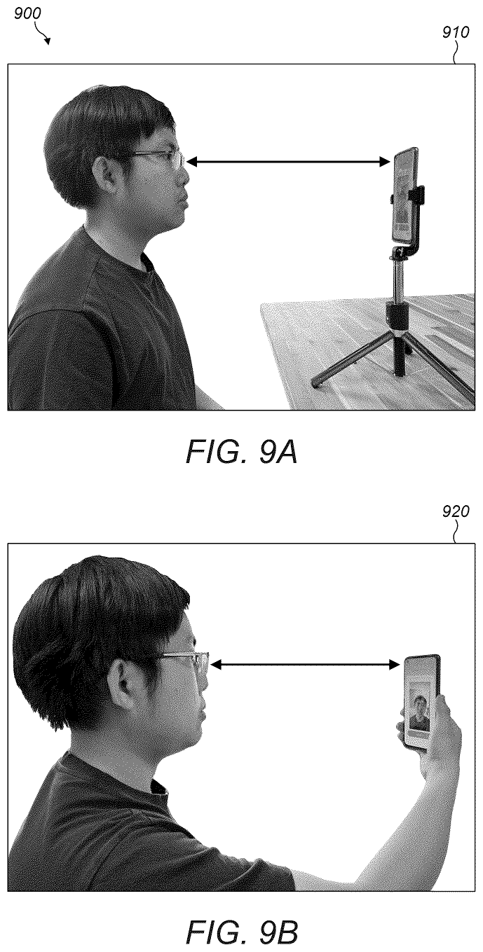

Turning to FIGS. 9A and 9B, shown is a spread of a picture 910 of how to hold a phone without a tripod cases and a picture 920 of how to hold a phone with a tripod case.
Please make sure the phone camera is at eye level (as indicated by the arrow)
Set the distance in such a way that the rectangle around your face is green (not red) and wait in that position until the countdown timer ends
For all next tasks, please try to keep your head in roughly this position
Please do not position your phone lower than your face.

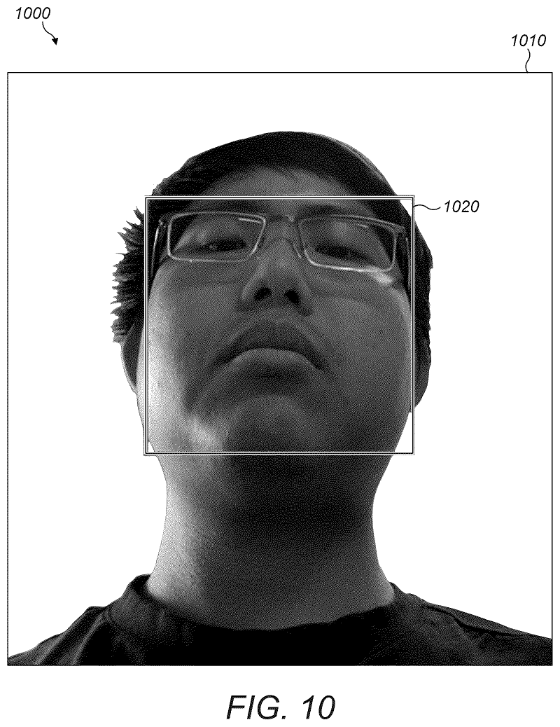

Turning to FIG. 10, shown is a spread 1000 of a photo 1010 showing a capture 1020 of how not to position the phone.

Task 2. Gaze Tracking.
Please aim to follow the circle by only using your eyes while keeping your head as still as possible.

Task 3. Read Aloud.
With the phone held as described before, please use your gaze to read the text presented to you. You will be asked to read multiple pages of text.

Task 4. Picture Description.
With the phone held as described before, please talk about the picture you are shown for at least 40 seconds (while the picture is shown on the screen).
Feel free to speak about any details that you might notice or about how it makes you feel.

Please try not to get very close to the screen in an attempt to see more details, also please try not to zoom as this is not possible.

If a user is silent for a while, the system may ask a follow up question to help the user, for example ‘how does this make you feel’ or ‘do you like the picture’

Task 5. Expression Mimicry.
With the phone held as described before, please mimic the facial expression displayed by the person in the video at the same time as they are doing it.
In order to get familiar with the facial expressions, please have a look at the 4 videos

In order to extract the facial landmarks, gaze vectors, action unit intensities and head pose from the videos, each of them were processed using an SDK. The output is a .csv file where each line contains all the extracted features for each of the frames in the video.

IV. TASK EVALUATION RESULTS

A. Gaze Tracking

From face video recordings collected in this task, we analyzed two key fatigue markers: 1. Saccades extracted from gaze vector angles and 2. Blink patterns derived from eyelid landmarks. These two markers are widely studied in the literature of fatigue measures from facial features [2][3][4].

1. Saccades

Saccades are abrupt temporal transitions in eye gaze directions. Saccadic velocity is a well-known fatigue feature in the literature [3][4]. In this analysis, we computed saccadic angular velocity (degrees/second) from yaw and pitch gaze angles estimated from face videos using the SDK.

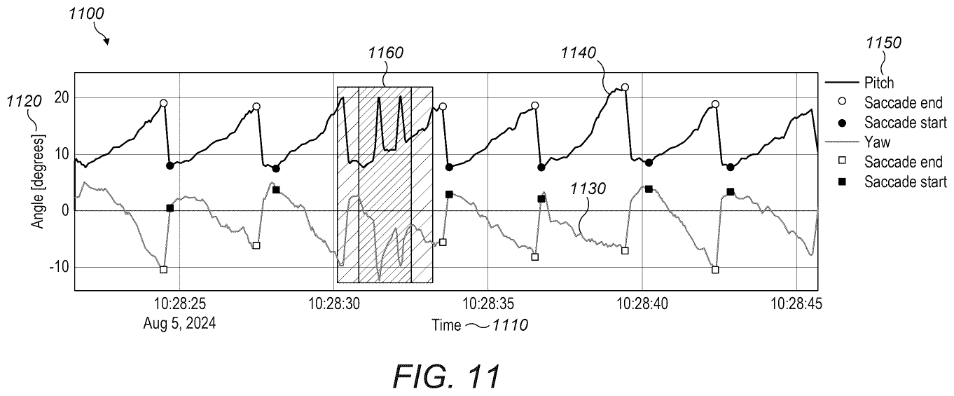

Turning to FIG. 11, shown in a graph 1100 with time 1110 as the x-axis and angle in degrees 1120 as the y-axis with a saccade pitch plot 1140 and a saccade yaw plot 1130 and a key 1150. The shaded region 1160 represents data that is ignored because of a blink.

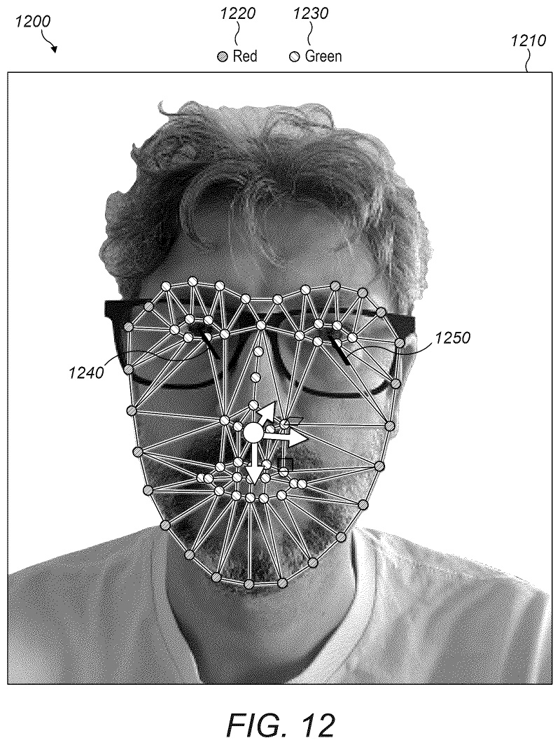

Turning to FIG. 12, shown is a schematic 1200 of a face overlay 1210 with red dots 1220 overlying outer face features and green dots 1230 overlaying inner face features. Vectors 1240 1250 are overlaid over the eyes.

FIG. 11 shows a sample of the data collected during the Gaze tracking task. The extracted saccadic segments from the gaze angles capture the sudden changes in dot locations presented in the Gaze Tracking task. This clearly shows that it is possible to reliably measure saccadic velocity from the data collected using the Gaze Tracking task.

One source of noise in saccadic features is eye blink. Blinks introduce noise in the estimated gaze directions by causing temporary occlusions of the inner eye appearance. We handle this issue by filtering out such temporal segments of gaze directions as shown in the shaded region 1160, with the help of our blink detection algorithm.

The two traces presented in FIG. 11 are of yaw and pitch angles of the gaze vectors outputted by the SDK, represented by the vectors 1240 1250 overlaid on the eyes in the image in FIG. 12. The abrupt changes represent the saccades occurring when a participant moves their gaze from the bottom of the screen to the top. The start and end of the saccades are represented by the circles and squares. The shaded region 1160 represents the data that is ignored because of blinks. Using both pitch and yaw, the angle between the vector at the start and end of the saccade is computed as well as the time duration; this allows us to extract saccade velocity, expressed in degrees/second.

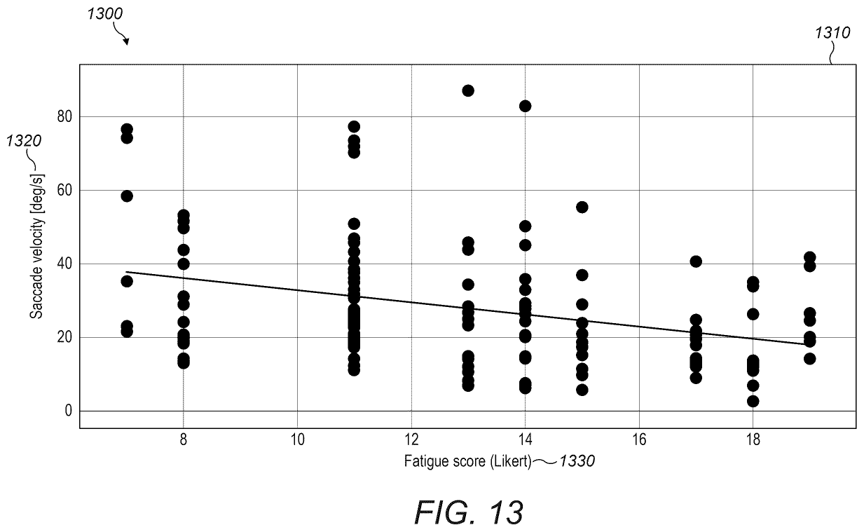

Turning to FIG. 13, shown in a graph 1300 of a plot 1310 of fatigue score with a Likert scale 1330 as the x-axis and saccade velocity in degrees/second 1320 as the y-axis.

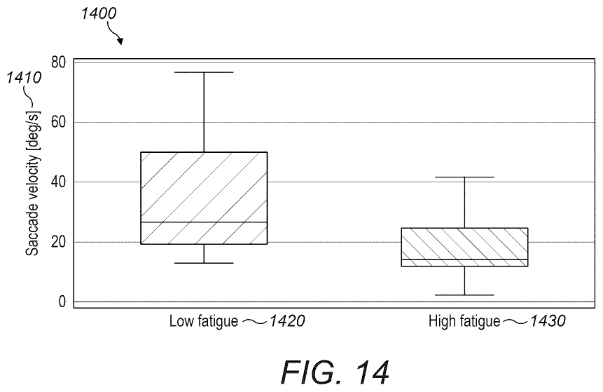

Turning to FIG. 14, shown is a boxplot 1400 of saccade velocity in degrees/second 1410 for low fatigue levels 1420 and high fatigue levels 1430.

Boxplots use boxes and lines to depict the distributions of one or more groups of numeric data by quartiles. Box limits indicate the range of the central 50% of the data, with a central line marking the median value. Lines extend from each box to capture the range of the remaining data. The top line is the beginning of the first quartile and bottom line is the ending of the fourth quartile. Dots placed past line edges indicate outliers.

The relationship in FIG. 13 is represented as a comparison between low and high fatigue levels in the boxplot in FIG. 14. These results clearly show that the measured saccadic velocity values from the GazeTracking task are noticeably distinct between the high fatigue level 1430 and low fatigue level 1420.

2. Blinks

Blinks are brief events of eyelid closures, which have been extensively explored in the literature of fatigue characterization [2][4]. In this analysis, we computed two standard blink parameters: blink rate and blink duration. The robustness of the eye landmark detection module in the SDK coupled with a blink power computation algorithm allowed us to automatically first detect blink occurrences and then extract blink rate and duration parameters.

The following is a description of blink phases based on its power curve characteristics:

Phase 1 starts as the eyelid closure begins, and ends when the total power exerted by the eyelid muscles reaches maximum.

Phase 2 is between the end of phase 1 and the moment the eyelid muscles reach the maximum closure velocity.

Phase 3 begins as the eyelid starts slowing down, and ends the moment its power curve reaches the minimum.

Phase 4 is between the end of phase 3 and the moment the eyelid closure phase ends completely and its opening phase begins.

Phase 5 starts as the eyelid moves upward and ends when the total power developed by the muscles reaches a local maximum.

Phase 6 is between the end of phase 5 and the moment the eyelid reaches a maximum velocity and its power curve reaches zero.

Phase 7 is between the end of phase 6 and the moment the eyelid opening begins slowing down and its power curve reaches a local minimum.

Phase 8 is between the end of phase 7 and the eyelid is fully open and its power curve reaches zero.

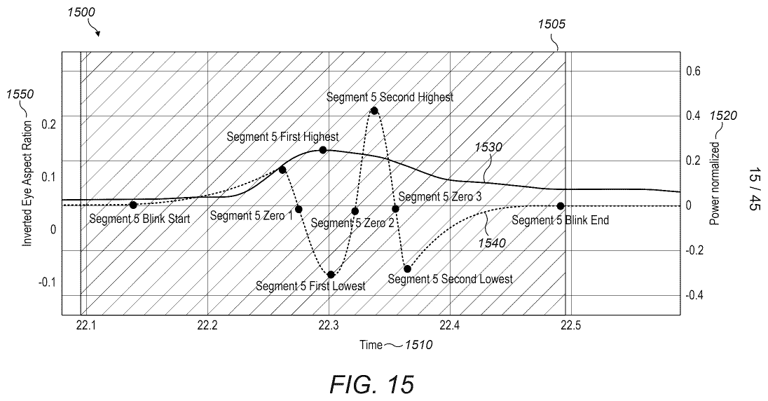

Turning to FIG. 15, shown is a graph 1500 of a plot 1505 with time 1510 as the x-axis, inverted eye aspect ratio 1550 as the left y-axis, and normalized power 1520 as the right y-axis. The solid line 1530 is tied to the left y-axis; the dashed line 1540 is tied to the right axis.

As shown in FIG. 15, Phase 3 starts after the eyelid muscles have reached maximum closure velocity and it represents a breaking phase, the power developed here is negative. This is represented on the graphs in FIG. 15 between Segment 5 Zero 1 and Segment 5 First lowest. (The “5” in Segment 5 is only a designation of a data set portion.)

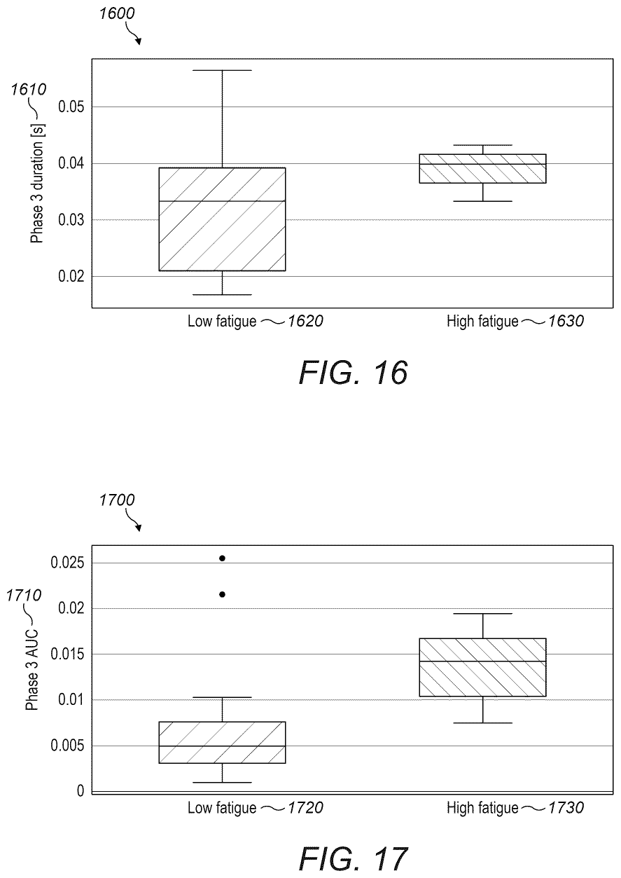

Turning to FIG. 16, shown is a boxplot 1600 of blink phase 3 duration in seconds 1610 for low fatigue levels 1620 and high fatigue levels 1630.

Turning to FIG. 17, shown is a boxplot 1700 of blink phase 3 area under the curve (AUC) 1710 for low fatigue levels 1720 and high fatigue levels 1730. AUC is equivalent to a measure of energy in phase 3. The area under the curve indicates a clear difference between the data collected in low vs high fatigue conditions.

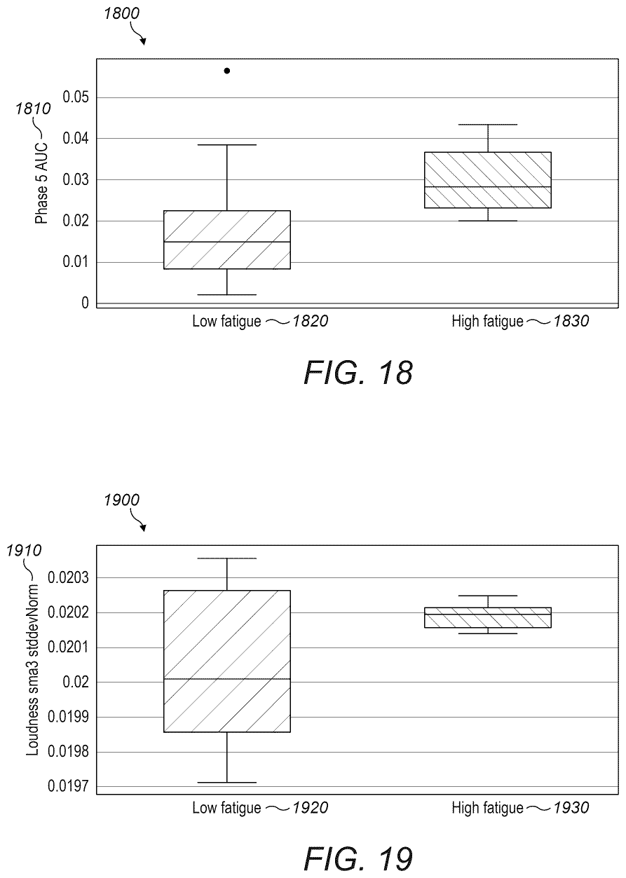

Turning to FIG. 18, shown is a boxplot 1800 of blink phase 5 area under the curve (AUC) 1810 for low fatigue levels 1820 and high fatigue levels 1830.

Phase 5, this is the opening phase, from when the eyelids start opening until the eyelids are fully opened. This also shows a clear difference between the low and high fatigue conditions.

The results presented above indicate that the gaze tracking task is a very good candidate for the fatigue app. Saccade velocity and eye closure features appear to be sensitive to changes in fatigue level and this task allows us to induce saccades which are relatively easy to detect and measure. In contrast, eye closure features are not induced by the app and can happen naturally.

B. Read Aloud

Speech features are known to contain information about fatigue levels [6]. To evaluate how well the Read Aloud task can elicit fatigue levels in the captured audio recordings, in this analysis we used two simple speech-based features: 1. Variability in speech loudness (measured in sma3 stddev/Norm); and 2. Speech articulation rate (number of pauses).

Turning to FIG. 19, shown is a boxplot 1900 of speech loudness (measured in sma3 stddev/Norm) 1910 for low fatigue levels 1920 and high fatigue levels 1930. “sma3 stddev/Norm audio” refers to concepts in audio signal processing, specifically a Simple Moving Average (SMA) with a window of 3 samples (SMA3) of normalized (Norm) audio and then taking the Standard Deviation of the loudness (StdDev).

In FIG. 19, we can observe that for the high fatigue samples, the median loudness is higher and also the data is less spread than in the low fatigue samples.

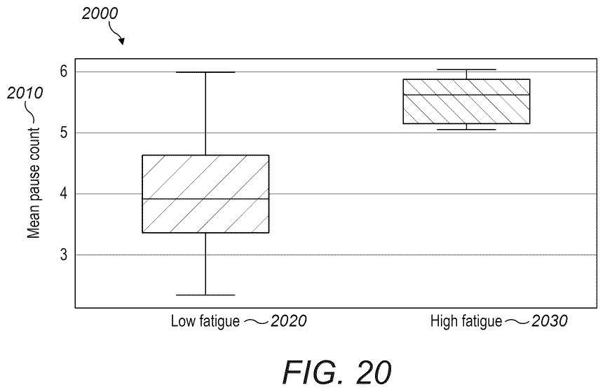

Turning to FIG. 20, shown is a boxplot 2000 of mean pause count 2010 for low fatigue levels 2020 and high fatigue levels 2030. Here we can observe that the median of the mean pause count is higher for the high fatigue samples.

1. Saccades

In the case of the read aloud task, the sampled saccades are induced by the participants moving their gaze from the end of one row of text to the beginning of another.

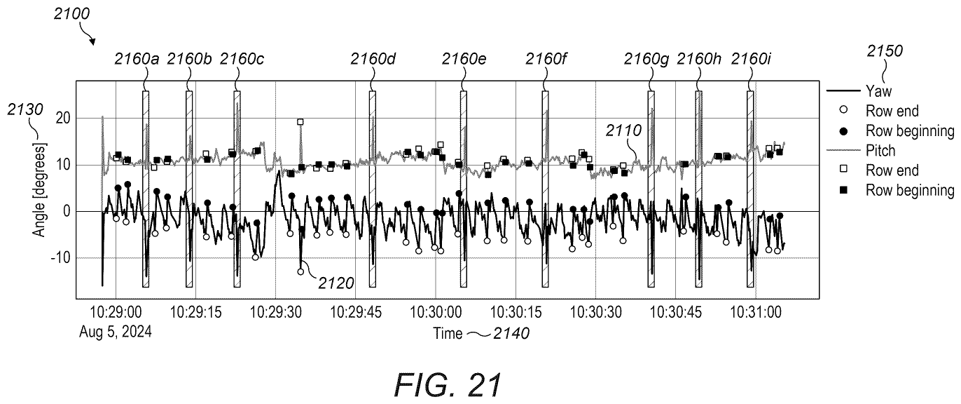

Turning to FIG. 21, shown in a graph 2100 with time 2140 as the x-axis and angle in degrees 2130 as the y-axis with a saccade yaw plot 2110 and a saccade pitch plot 2120 and a key 2150. The shaded regions 2160a-2160i represent data that is ignored because of a blink.

The image in FIG. 21 shows an example of yaw and pitch data with the saccade start (row end) and end (row beginning) indicated by the hollow and filled markers for the read aloud task. The shaded areas are identified blinks and were discarded. Same as for the previous task, we compute saccade velocity as one of the features based on these identified start and end points.

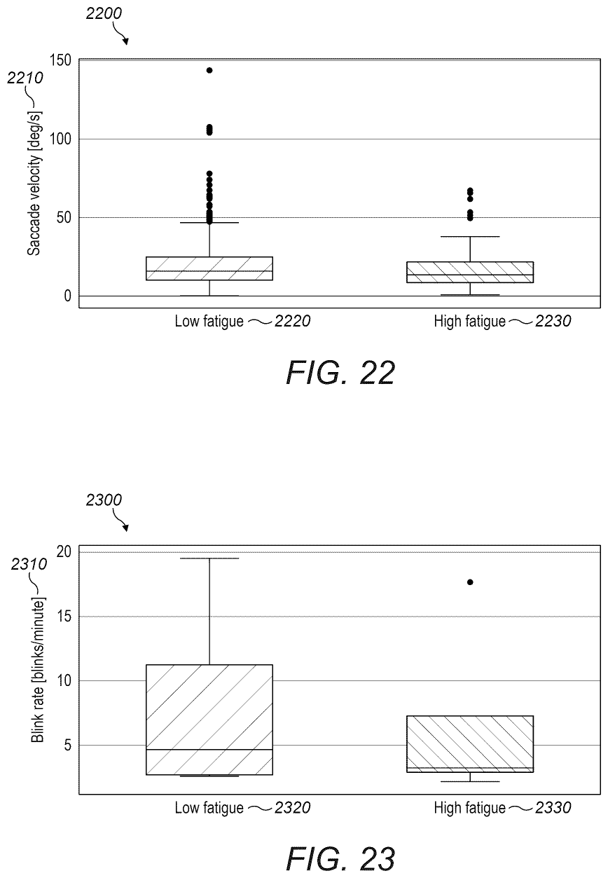

Turning to FIG. 22, shown is a boxplot 2200 of saccade velocity in degrees/second 2210 for low fatigue levels 2220 and high fatigue levels 2230.

The graph in FIG. 22 presents two boxplots comparing saccade velocity in the read aloud task for low and high fatigue levels.

2. Blink Features

Blink features for the read aloud task did not show major differences between the low and high fatigue conditions.

Turning to FIG. 23, shown is a boxplot 2300 of blink rate in blinks/minute 2310 for low fatigue levels 2320 and high fatigue levels 2330.

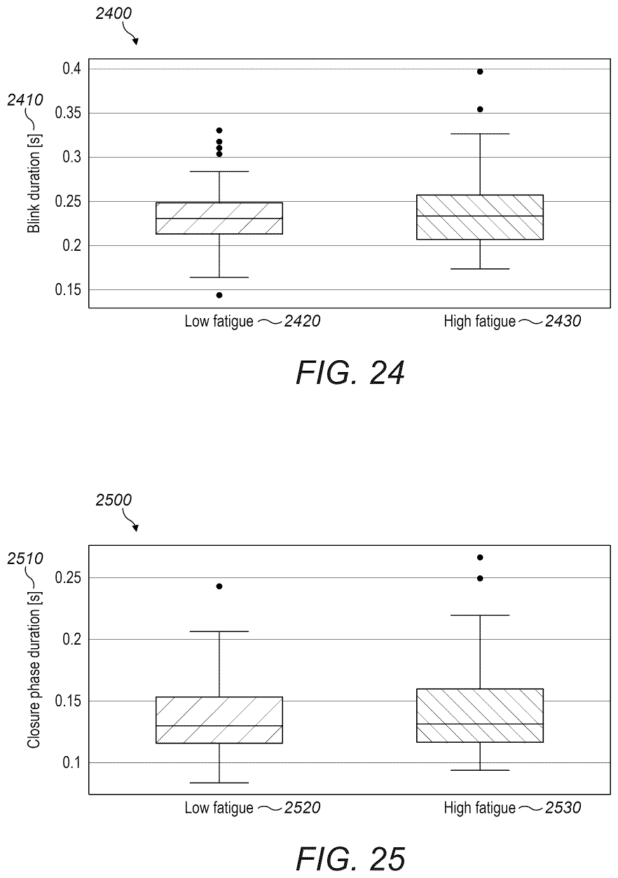

Turning to FIG. 24, shown is a boxplot 2400 of blink duration in seconds 2410 for low fatigue levels 2420 and high fatigue levels 2430.

Turning to FIG. 25, shown is a boxplot 2500 of closure phase duration in seconds 2510 for low fatigue levels 2520 and high fatigue levels 2530.

While for this sample of data, the blink and gaze features do not seem to be considerably different when sampled in low or high fatigue conditions, we do notice differences in terms of audio features sampled in the two conditions.

C. Picture Description

As this task involves visually scanning the contents of an image and describing them for at least 40 seconds, we considered the following four features extracted from the audiovisual recordings: 1. Facial muscle movements; 2. Facial expression intensity; 3. Speech features; and 4. Blinks.

1. Facial muscle movements

Facial Action Coding System (FACS) describes fine-grained facial muscle movements in terms of Action Units (AUs) and their intensity values. Each AU describes atomic changes in facial muscle movements on the scale of [0, 5]. In this analysis, we compare the maximum intensity values different AUs can reach between low and high fatigue examples.

AU main codes include:
0 Neutral face
1 Inner brow raiser
2 Outer brow raiser
4 Brow lowerer
5 Upper lid raiser
6 Cheek raiser
7 Lid tightener
8 Lips toward each other
9 Nose wrinkler
10 Upper lip raiser
11 Nasolabial deepener
12 Lip corner puller
13 Sharp lip puller
14 Dimpler
15 Lip corner depressor
16 Lower lip depressor
17 Chin raiser
18 Lip pucker
19 Tongue show
20 Lip stretcher
21 Neck tightener
22 Lip funneler
23 Lip tightener
24 Lip pressor
25 Lips part
26 Jaw drop
27 Mouth stretch
28 Lip suck
AU head movement codes include:
51 Head turn left
52 Head turn right
53 Head up
54 Head down
55 Head tilt left
M55 Head tilt left
56 Head tilt right
M56 Head tilt right
57 Head forward
M57 Head thrust forward
58 Head back
M59 Head shake up and down
M60 Head shake side to side
M83 Head upward and to the side
AU eye movement codes include:
61 Eyes turn left
M61 Eyes left
62 Eyes turn right
M62 Eyes right
63 Eyes up
64 Eyes down
65 Walleye
66 Cross-eye
M68 Upward rolling of eyes
69 Eyes positioned to look at other person
M69 Head or eyes look at other person
AU visibility codes include:
70 Brows and forehead not visible
71 Eyes not visible
72 Lower face not visible
73 Entire face not visible
74 Unscorable
AU gross behavior codes include:
29 Jaw thrust
30 Jaw sideways
31 Jaw clencher
32 [Lip] bite
33 [Cheek] blow
34 [Cheek] puff
35 [Cheek] suck
36 [Tongue] bulge
37 Lip wipe
38 Nostril dilator
39 Nostril compressor
40 Sniff
41 Lid droop
42 Slit
43 Eyes closed
44 Squint
45 Blink
46 Wink
50 Speech
80 Swallow
81 Chewing
82 Shoulder shrug
84 Head shake back and forth
85 Head nod up and down
91 Flash
92 Partial flash
97 Shiver/tremble
98 Fast up-down look

Due to the difficulty of automatically measuring AU intensities in in-the-wild face video data, there is a very limited amount of existing work [5] on using AUs as direct measures of fatigue. In this analysis, since eye lid features such as blink rates and durations and gaze features cover the key fatigue markers in the upper half of the face, we focus on seven AUs in the lower half of the face.

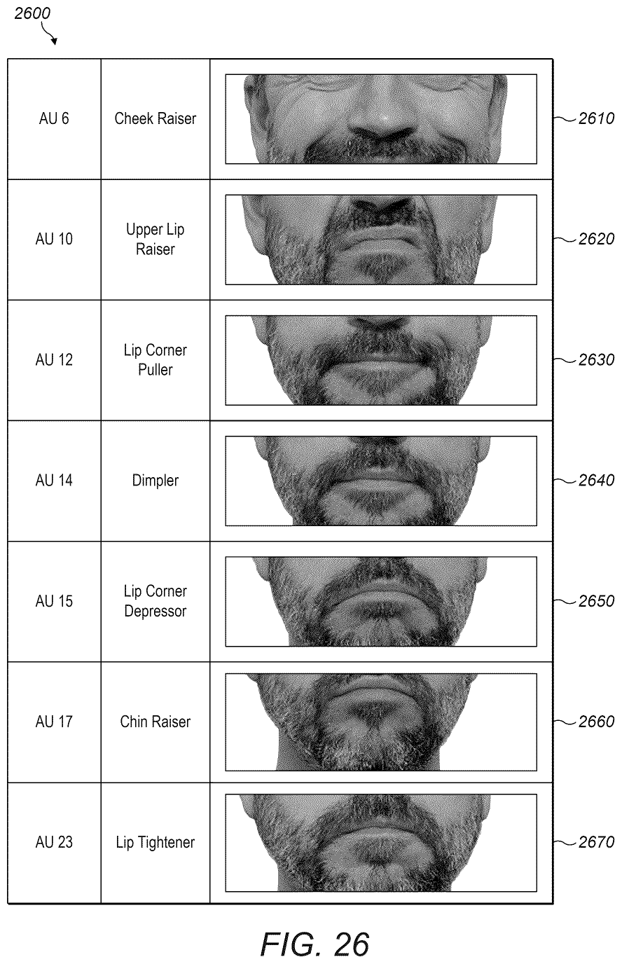

Turning to FIG. 26, shown is a spread of 7 action units of the lower half of the face analyzed in this study:
AU 6 (Cheek Raiser) 2610;
AU 10 (Upper Lip Raiser) 2620;
AU 12 (Lip Corner Puller) 2630;
AU 14 (Dimpler) 2640;
AU 15 (Lip Corner Depressor) 2650;
AU 17 (Chin Raiser) 2660; and
AU 23 (Lip Tightener) 2670.

2. Facial expression intensity

We first extracted the raw intensity estimates of these seven AUs and then derived AU-wise maximum intensity values across the task, for both low and high fatigue cases. The same raw AU intensity values can be used for extracting more advanced features such as fine-grained temporal dynamics of facial movements and co-occurrence patterns of AUs, etc.

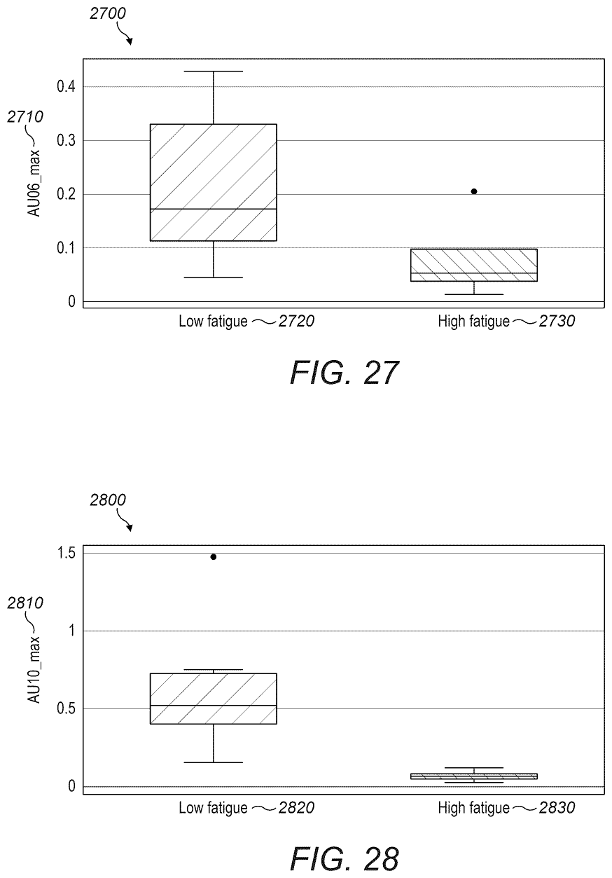

Turning to FIG. 27, shown is a boxplot 2700 of AU 6 (Cheek Raiser) 2710 for low fatigue levels 2720 and high fatigue levels 2730.

Turning to FIG. 28, shown is a boxplot 2800 of AU 10 (Upper Lip Raiser) 2810 for low fatigue levels 2820 and high fatigue levels 2830.

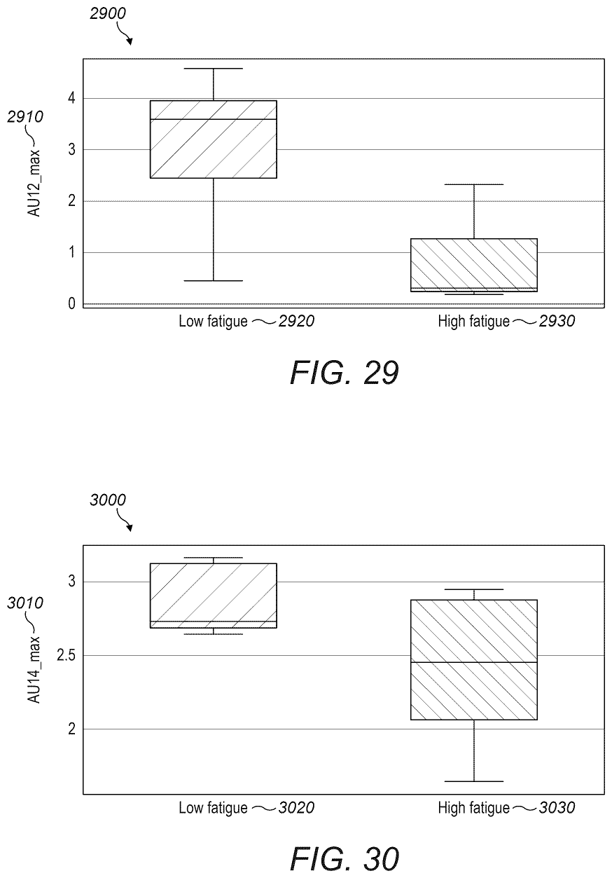

Turning to FIG. 29, shown is a boxplot 2900 of AU 12 (Lip Corner Puller) 2910 for low fatigue levels 2920 and high fatigue levels 2930.

Turning to FIG. 30, shown is a boxplot 3000 of AU 14 (Dimpler) 3010 for low fatigue levels 3020 and high fatigue levels 3030.

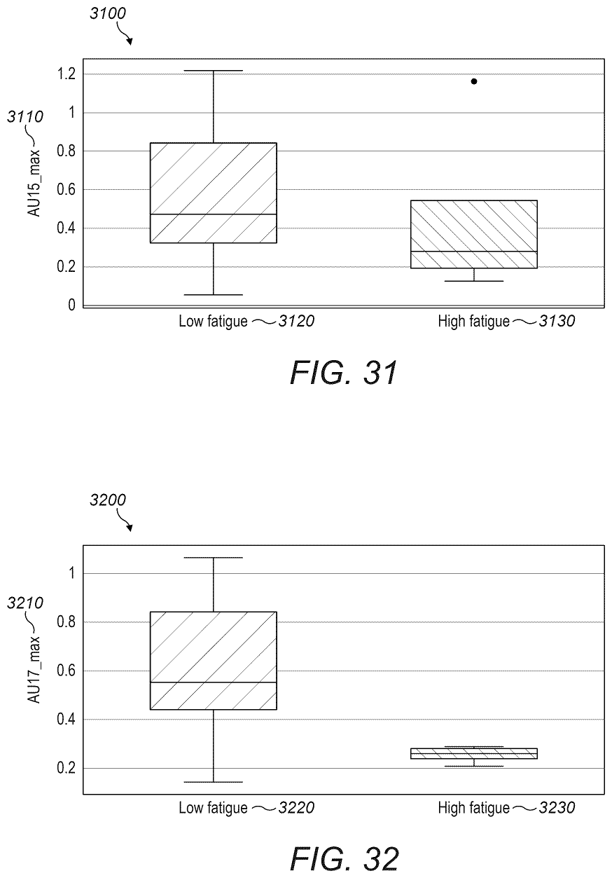

Turning to FIG. 31, shown is a boxplot 3100 of AU 15 (Lip Corner Depressor) 3110 for low fatigue levels 3120 and high fatigue levels 3130.

Turning to FIG. 32, shown is a boxplot 3200 of AU 17 (Chin Raiser) 3210 for low fatigue levels 3220 and high fatigue levels 3230.

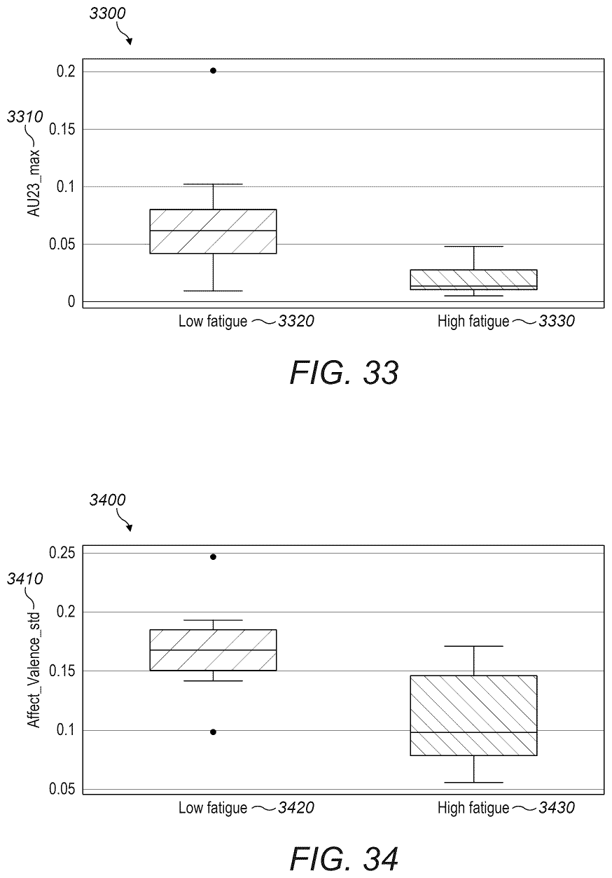

Turning to FIG. 33, shown is a boxplot 3300 of AU 23 (Lip Tightener) 3310 for low fatigue levels 3320 and high fatigue levels 3330.

As shown in FIGS. 27, 28, 29, 30, 31, 32, and 33, box plots across all seven AUs the median feature values of low and high fatigue conditions differ by noticeably margins. AU 12 (lip corner puller) shows the highest margin and AU 23 (lip tightener) has the lowest margin. Overall, these results show that the AU intensity features captured from the Picture Description task indicate noticeable differences between low and fatigue classes.

Facial expression variability refers to the overall variance of expressed emotions in facial displays across the task. Leveraging the robustness and accuracy of the Dimensional Emotion recognition model [7] integrated into the SDK, we measured the temporal variance of expressed emotion values across the frames captured in this task.

To measure the expressed emotion values, here we used Emotional Valence dimension [7] as it captures how positive or negative an emotion is on a continuous scale in the range [−1, 1]. We first extracted per-frame valence predictions from raw face video recordings and then computed standard deviation of valence predictions across the video.

Turning to FIG. 34, shown is a boxplot 3400 of valance standard deviation 3410 for low fatigue levels 3420 and high fatigue levels 3430.

FIG. 34 compares the valence standard deviation values of low and high fatigue conditions. It clearly shows that compared to low fatigue cases high fatigue cases show less valence variability. This result is aligned with the intuition that fatigue may negatively impact the levels of facial expressivity.

3. Speech Features

Speech Features considered in this task are same as that in the Read Aloud task: 1. Variability in speech loudness; and 2. Speech articulation rate (average number of pauses).

It is important to note that the speech data collected in the Read Aloud task is scripted whereas it is spontaneous speech in the case of Picture Description task. Due to this difference, the extracted speech features in this task are likely to show some deviations from the results of the Read Aloud task.

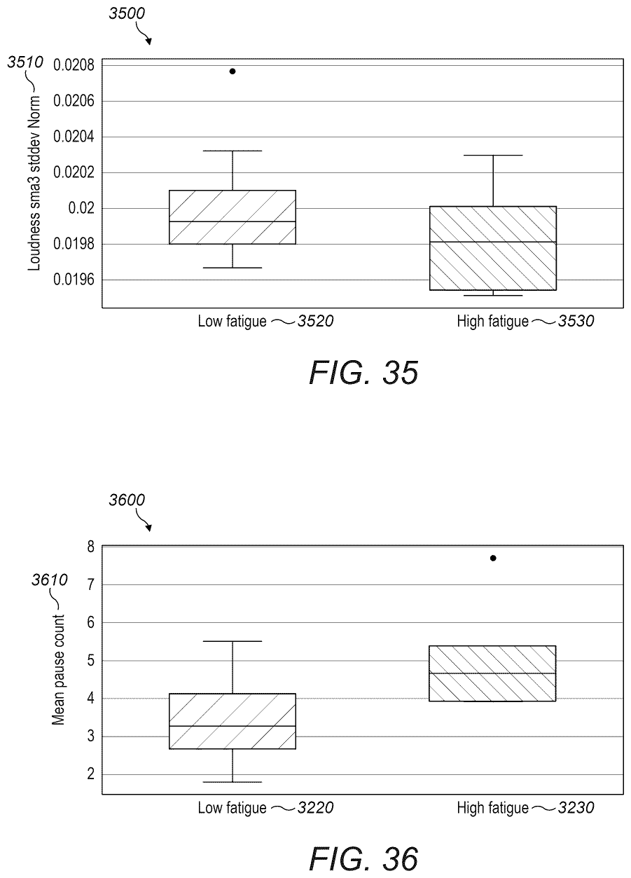

Turning to FIG. 35, shown is a boxplot 3500 of speech loudness (measured in sma3 stddev/Norm) 3510 for low fatigue levels 3520 and high fatigue levels 3530.

Turning to FIG. 36, shown is a boxplot 3600 of mean pause count 3610 for low fatigue levels 3620 and high fatigue levels 3630.

The boxplots shown in FIGS. 35 and 36 compare these two features for low and high fatigue conditions. These results indicate that compared to the loudness variability feature, the speech articulation rate, i.e., mean pause count looks relatively more distinct between high and low fatigue cases.

4. Blinks

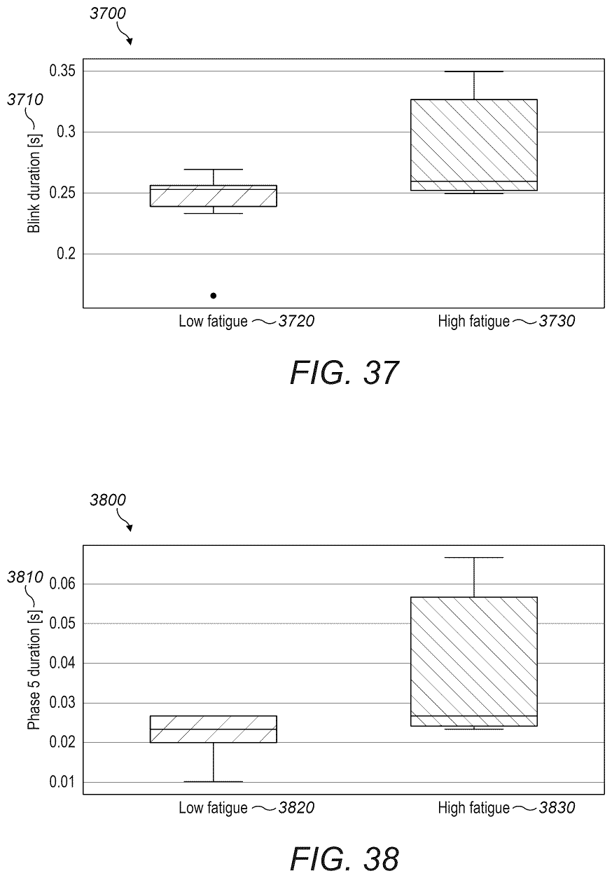

Turning to FIG. 37, shown is a boxplot 3700 of blink duration in seconds 3710 for low fatigue levels 3720 and high fatigue levels 3730.

Turning to FIG. 38, shown is a boxplot 3800 of blink phase 5 duration in seconds 3810 for low fatigue levels 3820 and high fatigue levels 3830.

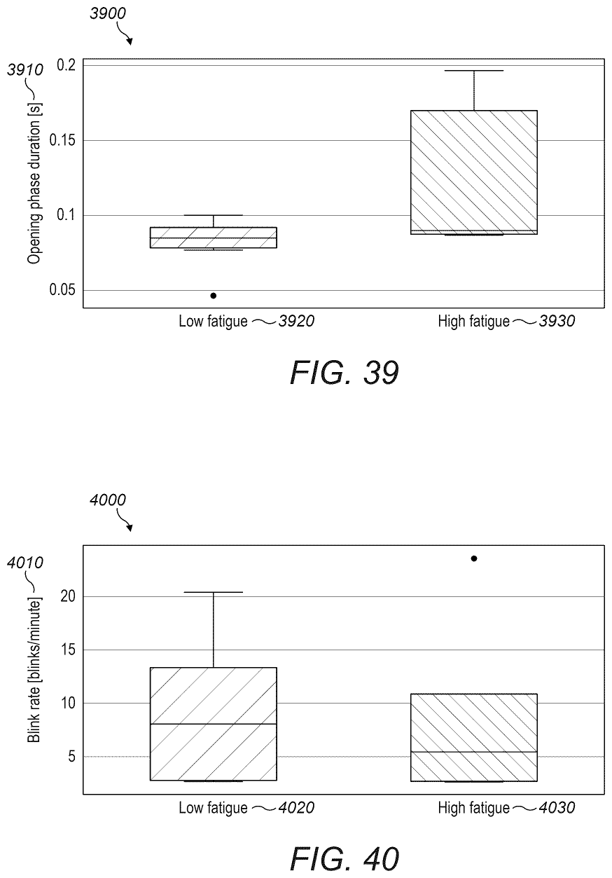

Turning to FIG. 39, shown is a boxplot 3900 of opening phase duration in seconds 3910 for low fatigue levels 3920 and high fatigue levels 3930.

Turning to FIG. 40, shown is a boxplot 4000 of blink rate in blinks per minute 4010 for low fatigue levels 4020 and high fatigue levels 4030.

For the Picture description task, FIGS. 37, 38, 39, and 40 show the differences between low and high fatigue for a few of the blink feature metrics that were computed. We can observe some differences for blink duration, phase 5 duration of the blinks (opening phase when maximum power is observed), and opening phase duration. For blink rate, no noticeable difference was observed.

For the voice features, the mean pause count seems to show a clear difference between the samples collected in the low and high fatigue conditions, similar to what was observed in the Read Aloud task. The activation of the action units also show clear differences, in general lower activation is observed in the high fatigue condition. Blink features are also considerably different between the two conditions, except for blink rate which is similar. While it is similar to the Read Aloud task in terms of voice features, this task has the added element of also engaging more complex thought processes needed for the interpretation of the image and relationships between the elements as well as language production in an unguided way, all of these can be impacted by fatigue.

D. Expression Mimicry

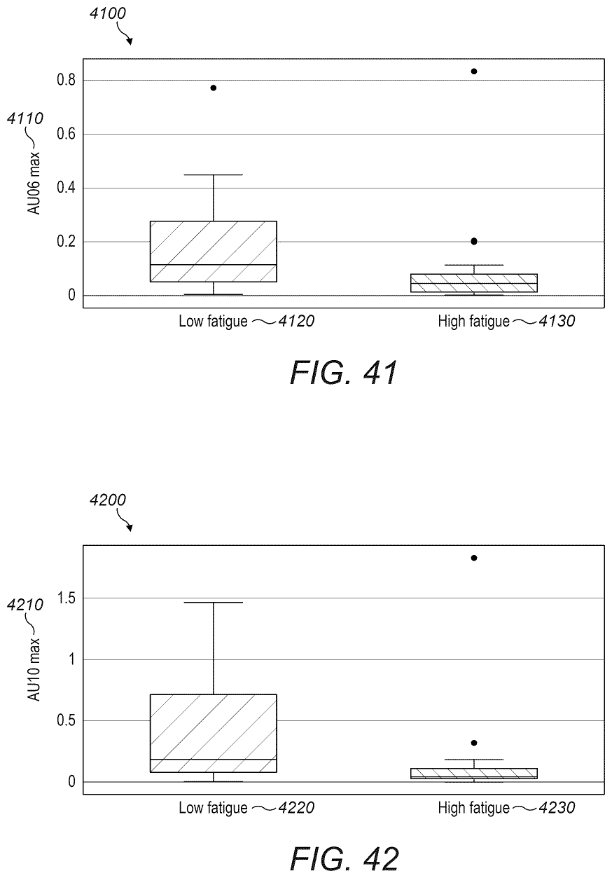

Turning to FIG. 41, shown is a boxplot 4100 of AU 06 (cheek raiser) 4110 for low fatigue levels 4120 and high fatigue levels 4130.

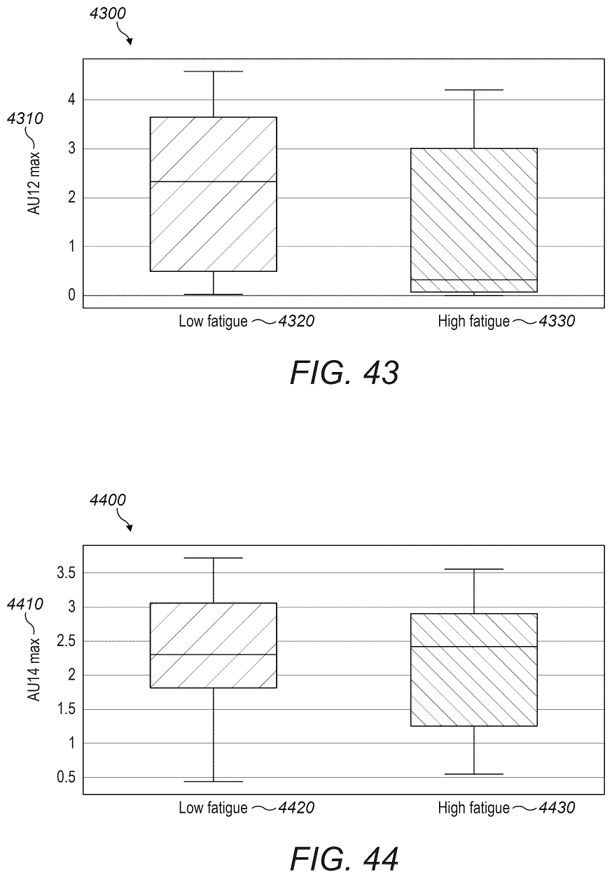

Turning to FIG. 42, shown is a boxplot 4200 of AU 10 (upper lip raiser) 4210 for low fatigue levels 4220 and high fatigue levels 4230.

Turning to FIG. 43, shown is a boxplot 4300 of AU 12 (lip corner puller) 4310 for low fatigue levels 4320 and high fatigue levels 4330.

Turning to FIG. 44, shown is a boxplot 4400 of AU 14 (dimpler) 4410 for low fatigue levels 4420 and high fatigue levels 4430.

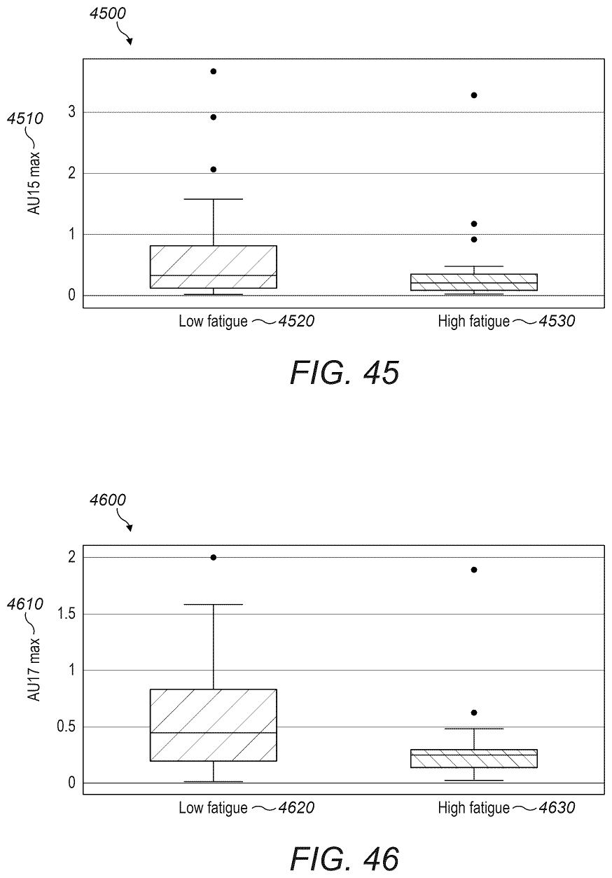

Turning to FIG. 45, shown is a boxplot 4500 of AU 15 (lip corner depressor) 4510 for low fatigue levels 4520 and high fatigue levels 4530.

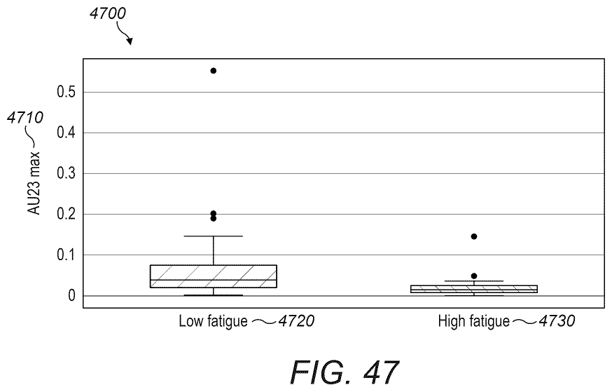

Turning to FIG. 46, shown is a boxplot 4600 of AU 17 (chin raiser) 4610 for low fatigue levels 4620 and high fatigue levels 4630.

Turning to FIG. 47, shown is a boxplot 4700 of AU 23 (lip tightener) 4710 for low fatigue levels 4720 and high fatigue levels 4730.

This guided facial expression mimicry task intends to induce a full range of facial muscle movements by asking participants to mimic the facial expressions shown in the stimuli videos. Similar to the Picture Description data analysis, here we focused on evaluating seven AU features in the lower half of the face, since blink and saccade features captured in the gaze tracking and read aloud tasks already cover eye related muscle movements.

We first extracted the raw intensity values for the seven AUs from the face recordings captured in this mimicry task. Then we computed the maximum AU intensity values for both low and high fatigue classes. AU-wise comparisons of these features for low and high fatigue conditions are shown below:

As shown in the box plots in FIGS. 41, 42, 43, 44, 45, 46, and 47, the AU features extracted here do not show any noticeable differences, unlike in the case of Picture Description. Except for AUs 6 and 10, all other AU features have a significant overlap between low and high fatigue classes. These results indicate that the face videos captured in the Expression Mimicry task seem to be less useful for eliciting clearly measurable fatigue markers in facial muscle movements.

It is also worth noting that the face video recordings obtained from the Expression Mimicry task are relatively short-duration clips (<5 seconds). AU predictions tend to be less accurate in such short duration video clips due to the lack of sufficient temporal context for facial movement analysis. This may make it difficult to derive reliable AU intensity predictions from the SDK for fatigue characterizations from the Expression Mimicry data.

V. DEFINITIONS

This section provides the definitions of all the key fatigue markers used in developing our fatigue estimation approach.

A. Mean reaction time

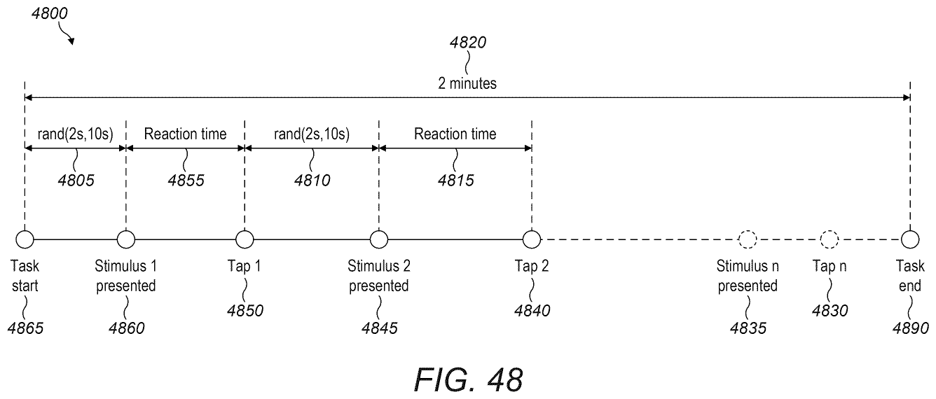

Turning to FIG. 48 is a timeline 4800 of a 2-minute task 4820 describing the task flow. The task starts at time zero 4865 and proceeds with a random time between 2 and 10 seconds 4805 until stimulus 1 is presented 4860. After a reaction time 4855, tap 1 occurs 4850 followed by a random time between 2 and 10 seconds 4810 until stimulus 2 is presented 4845. After a reaction time 4815, tap 2 occurs 4840. This process is repeated until stimulus n is presented 4835 and tap n occurs 4830 until task end 4890.

The mean reaction time metric is obtained using a psychomotor vigilance test. This task lasts for about 2 minutes and displays a stimulus at random inter stimulus intervals [randomized between 2 and 10 seconds]. Once the stimulus is presented, the participant has to tap on it as fast as possible. The time difference between stimulus presentation and participant tap on the screen constitutes the reaction time. This is repeated multiple times during the task and the reaction times are averaged, resulting in the mean reaction time metric. This task was inspired by [13].

B. Saccade velocity

Saccades are the eye movements that move the fovea rapidly from one point of interest to another. In our case this movement is estimated by rapid changes in the direction of the gaze vectors. Saccade velocity is therefore the speed of the eye movement during these rapid direction changes and it is expressed in degrees per second.

The evidence presented in this patent focuses on saccades induced in the following tasks:

Task 1. Tracking a smooth moving stimulus on a screen that changes location in a very brief time interval from the bottom of the screen to the top of the screen. See FIG. 3.

Task 2. Changing the gaze direction from the end of the line to the beginning of the next line in a read aloud task. See FIG. 4.

Examples of saccades detected in one of the object tracking tasks, presented in the pitch and yaw angles of the vectors-saccades are seen in FIG. 11 as large angular changes over brief time spans. The area overlaid with shading was excluded due to the presence of blinks that were filtered out. Blinking can be automatically filtered out with our facial muscle action detection. See FIG. 11

C. Blink features

Blink features are extracted from the eye aspect ratio signals, which represents the ratio between the vertical and horizontal distance as extracted from our automatically detected eye landmarks.

The blink phases are shown as follows:

Phase 1: the phase starting with the closure start and ending when the max power in closure phase is reached.

Phase 2: phase between max closure power and moment when the eyelid muscles stop working and eyelid reaches max closure velocity.

Phase 3: phase between the eyelid reaching max velocity closing up until reaching max power breaking the closure.

Phase 4: phase between the max power breaking in the closure phase and the moment the eye is fully closed.

Phase 5: the first opening phase, starting when the eye starts opening up until the eyelid reaches max power opening.

Phase 6: phase between max power opening and max velocity opening.

Phase 7: phase between max velocity opening and max power breaking during opening.

Phase 8: phase between max power breaking in the opening phase and the moment the eye is fully opened.

Based on the eye aspect ratio, we compute the normalized power per unit of mass developed by the eyelid muscles. This was based on the discussion in [8].

Shown in FIG. 15 is a sample of the actual data collected, the dashed line 1540 is the normalized power and it has a similar shape to the one presented in the paper above.

The features that are derived from this data include:

Total blink duration-in seconds.

Duration of each of the blink phases described by the blink start and each of the t_{N}p (N from 1 to 8) as described in the paper, resulting in 8 blink phases—in seconds.

Area under the curve for the previously defined 8 blink phases—dimensionless.

Total closure phase duration-in seconds.

Total opening phase duration-in seconds.

Blink rate (right and left).

Median saccade speed.

Standard deviation of saccade speeds.

D. Audio Features

Loudness sma3 stddevNorm: The coefficient of variation applied to the short-term smoothed estimate of perceived loudness.

Pitch_mean: The average perceived fundamental frequency (F0) of the voice over the analyzed segment.

Pitch_std: The standard deviation of the fundamental frequency (F0), indicating pitch variability or intonation range.

Loudness_mean: The average perceived intensity or volume level of the audio signal.

Loudness_std: The standard deviation of the perceived loudness, indicating the variation or dynamic range in volume.

Mean pause count: The mean duration of all the pauses detected between utterances or speech segments.

E. Voice VAD Features

The valence, arousal and dominance model was originally proposed by Albert Mehrabian and James Russell as a 2-dimensional circumplex model of valence and arousal, with dominance added later. It is the most common dimensional affect model. It describes every emotion as a point in a two-dimensional space, with the horizontal axis being Valence-how positive or negative you are feeling, and the vertical axis being arousal—how much energy you have, or sometimes interpreted as the intensity of the felt emotion. Arousal has therefore nothing to do with any feelings of sensual desire. Dominance describes how much a person feels in control of the situation. It is crucial to distinguish between emotions such as anger and fear, which largely overlap in the valence-arousal plane. Anger would have a high dominance value though, and fear a low dominance value.

To summarize the affective dimensions estimated from the voice signal, the following statistical features are computed for Valence (V), Arousal (A), and Dominance (D):

Voice_Median_V: The median value of the estimated valence over the analysis period.

Voice_Median_A: The median value of the estimated arousal over the analysis period.

Voice_Median_D: The median value of the estimated dominance over the analysis period.

Voice_Std_V: The standard deviation of the estimated valence, indicating its variability.

Voice_Std_A: The standard deviation of the estimated arousal, indicating its variability.

Voice_Std_D: The standard deviation of the estimated dominance, indicating its variability.

F. Action Unit Features

To characterize facial muscle dynamics potentially indicative of fatigue, we analyze Action Unit (AU) activation patterns over time. Key features extracted include:

the peak activation intensity (max),;

the rate at which activation accelerates (acc) and decelerates (dec); and

the total duration (dur) the AU remains active.

These features were extracted from 15 AUs detected by the SDK:
AU 01 Inner brow raiser
AU 02 Outer brow raiser
AU 04 Brow lowerer
AU 05 Upper lid raiser
AU 06 Cheek raiser
AU 07 Lid tightener
AU 09 Nose wrinkler
AU 10 Upper lip raiser
AU 12 Lip corner puller
AU 14 Dimpler
AU 15 Lip corner depressor
AU 17 Chin raiser
AU 23 Lip tightener
AU 25 Lips part
AU 45 Blink

G. Facial Emotion Features

We extract features capturing the typical level (median) and the amount of fluctuation (standard deviation) of valence and arousal, as estimated from face videos:

Face_Valence_Median: Represents the typical estimated valence level.

Face_Valence_Std: Quantifies the fluctuation in estimated valence.

Face_Arousal_Median: Represents the typical estimated arousal level.

Face_Arousal_Std: Quantifies the fluctuation in estimated arousal.

VI. EVALUATION RESULTS

The results are split into four sections:

1. System level fatigue evaluation results-demonstrating the capabilities of multiple metrics combined at system level and validation on a Depression dataset.

2. Validation of our fatigue markers in Chronic Liver Disease.

3. Individual fatigue feature evaluation results-showing the relationship between individual features and fatigue levels

4. In-car drowsiness detection validation-summarizing our drowsiness detection model's performance in a driving simulator as well as in real-world driving conditions.

A. System level validation results

These results are based on a dataset consisting of 998 audiovisual recordings collected from 738 unique participants. Each participant completed two structured interactive tasks implemented into a mobile application.

Mood Diary Task: Participants verbally describe their current mood, providing spontaneous and natural speech patterns.

Read-Aloud Task: Participants read a predefined text passage, ensuring consistency in spoken content across subjects.

Each audiovisual recording has self-reported PHQ-9 scores, a clinically validated tool for assessing depression severity. The PHQ-9 is a multipurpose instrument for screening, diagnosing, monitoring and measuring the severity of depression.

The PHQ-9 asks, “Over the last 2 weeks, how often have you been bothered by any of the following problems?” and presents the following 9 questions, each with 4 possible answers: Not at all (0), Several days (1), More than half the days (2), Nearly every day (3).

1. Little interest or pleasure in doing things.

2. Feeling down, depressed, or hopeless.

3. Trouble falling or staying asleep, or sleeping too much.

4. Feeling tired or having little energy.

5. Poor appetite or overeating.

6. Feeling bad about yourself—or that you are a failure or have let yourself or your family down.

7. Trouble concentrating on things, such as reading the newspaper or watching television.

8. Moving or speaking so slowly that other people could have noticed? Or the opposite—being so fidgety or restless that you have been moving around a lot more than usual.

9. Thoughts that you would be better off dead or of hurting yourself in some way.

PHQ-9 score obtained by adding score for each question (total points). Total scores of 5, 10, 15, and 20 represent cutpoints for mild, moderate, moderately severe and severe depression, respectively.

The ground truth that was used for fatigue is the 4 on the PHQ-9 questionnaire.

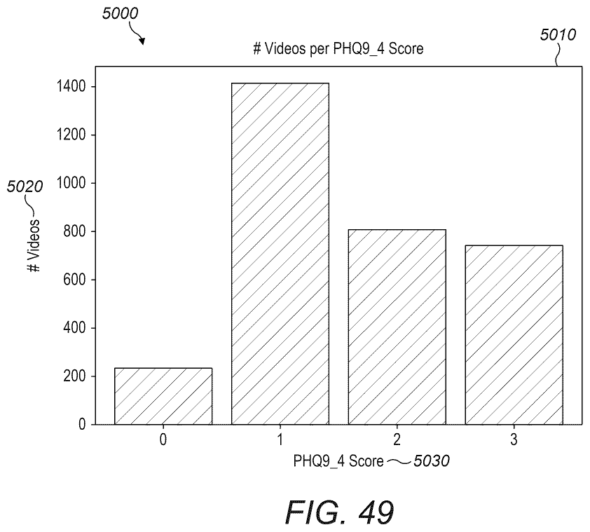

Turning to FIG. 49, shown is a graph 5000 of the number of videos per PHQ9_4 score 5010. The x-axis is the PHQ9_4 score 5030 and y-axis is the number of videos 5020. FIG. 49 thus shows the distribution of fatigue levels in the dataset.

The following fatigue features were used for the model training:

Oculometric features: Blink_Rate_Left; Blink_Rate_Right; Median_Saccade_Speed; Std_Dev_Saccade_Speed.

Personality trait scores as defined in [14]: Openness, Conscientiousness, Extraversion, Agreeableness, and Neuroticism.

Voice emotion features: Voice_Median_Valence; Voice_Median_Arousal; Voice_Median_Dominance; Voice_Std_Dev_Valence; Voice_Std_Dev_Arousal; Voice_Std_Dev_Dominance.

Voice acoustic features: Pitch_mean; Pitch_std_dev; Loudness_mean; Loudness_std_dev.

Facial muscle action features: Acceleration, deceleration, max intensity and duration features of AU01; AU02; AU04; AU05; AU06; AU07; AU09; AU10; AU12; AU14.

Facial emotion features: Face_Valence_Median; Face_Valence_Std; Face_Arousal_Median; Face_Arousal_Std

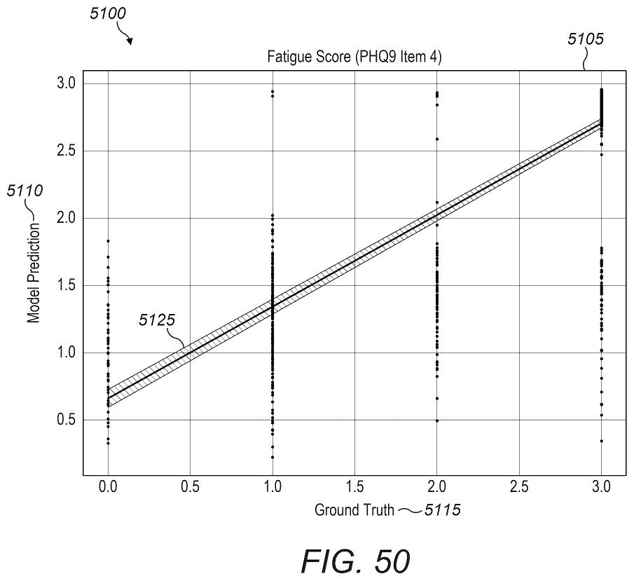

Turning to FIG. 50, shown is a graph 5100 of PHQ9_4 score 5105. The x-axis is the ground truth 5115 and the y-axis is the model prediction 5110. The variance of the ground truth and model prediction is shown in correlation line 5125.

After addressing the imbalances in the label distribution, the Pearson correlation coefficient of the fatigue labels outputted by the model with the ground truth labels was 0.812 and a root mean square error of 0.591 (out of a maximum of 4). This is shown as FIG. 50 by way of the correlation line 5125.

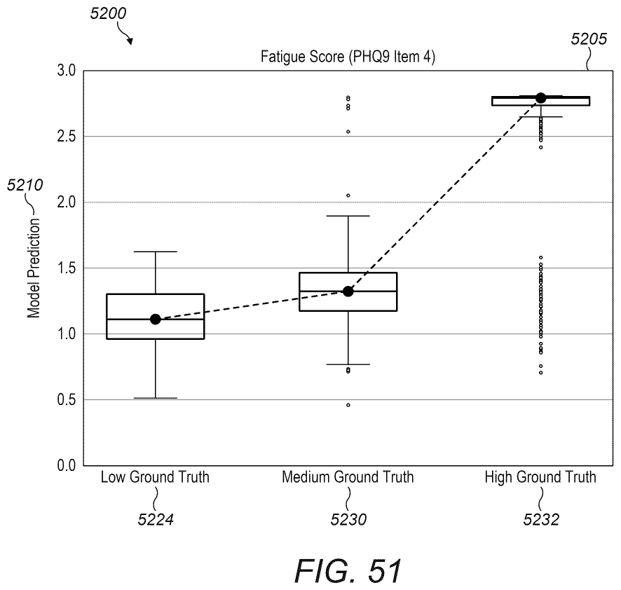

Turning to FIG. 51, shown is a boxplot 5200 of a PHQ9_4 score 5205 against model prediction 5210 in the y-axis for low ground truth 5224 (value 0), medium ground truth 5230 (value 1 or 2), and high ground truth 5232 (value 3). The model's predictions correlate strongly with the ground truth fatigue levels.

B. Individual Fatigue Feature Validation

These results were obtained from a smaller dataset consisting of 10 recordings from one participant. For this reason, they are presented separately and not as part of a model.

1. Gaze tracking task.

FIG. 13 shows how the saccade velocity tends to decrease as the fatigue score increases.

The fatigue levels were binned into two levels: high and low and we can visually see that the saccade velocity tends to be lower during the high fatigue recordings. This is shown in FIG. 14.

The graphs in FIGS. 17 and 18 show that the phase 3 and 5 area under curve metric tends to be lower for the low fatigue recordings.

2. Read aloud task.

For the read aloud task, FIG. 19 shows how the loudness sma3 stddevNorm has a much narrower distribution during the high fatigue intervals. For the same condition, FIG. 20 shows the pauses between utterances are consistently higher in the high fatigue condition.

3. Picture description task.

For the picture description condition, FIGS. 27, 28, 29, 30, 31, 32, 33 show the maximum action unit activation metrics for a number of action units and we can observe that some show differences between conditions.

FIG. 34 shows the standard deviation of the visually estimated valence for the high and low fatigue recordings.

FIG. 36 shows the mean pause duration appears to be generally higher in the high fatigue condition, same as in the read loud condition.

FIGS. 37, 38, 39 show blink durations as well as phase 5 duration and opening phase duration have generally higher values for the high fatigue condition.

C. Validation of Fatigue Markers in Chronic Liver Disease (CLD)

Fatigue markers are evaluated on real-world datasets, including audiovisual recordings from structured interactive tasks via a mobile app.

The evaluation dataset comprises a control group and individuals clinically diagnosed with CLD.

As an objective reference, we use reaction time duration from psychometric tasks. The reaction time task is a psychomotor vigilance test used to measure a person's alertness by recording their reaction time to a stimulus that is presented at random time intervals. This is one of the tools often used in fatigue research, considered the gold standard for objectively measuring sustained attention and detecting fatigue.

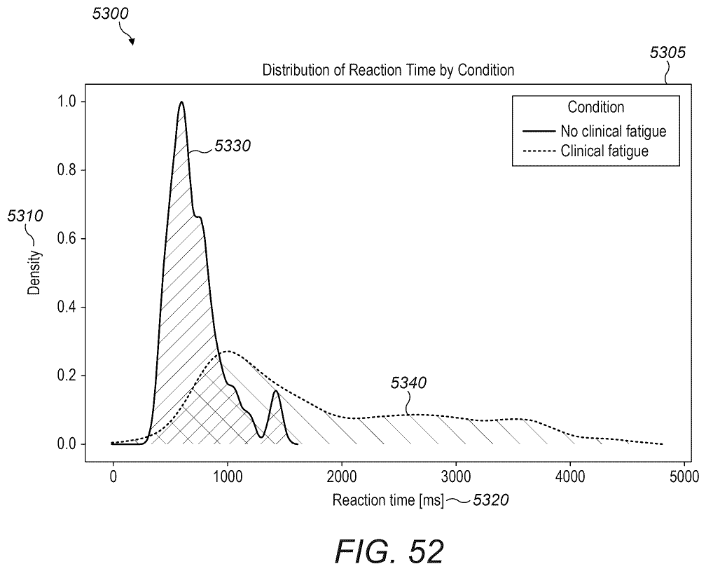

Turning to FIG. 52, shown is a graph 5300 of a plot 5305 of distribution reaction time by condition where the x-axis is reaction time in milliseconds 5320 and the y-axis is density of the data 5310. The no clinical fatigue condition 5330 is shown under the solid line and the clinical fatigue condition 5340 is shown under the dashed line. The density of the data 5310 is set such that the area under the curve of each condition is set to 1.

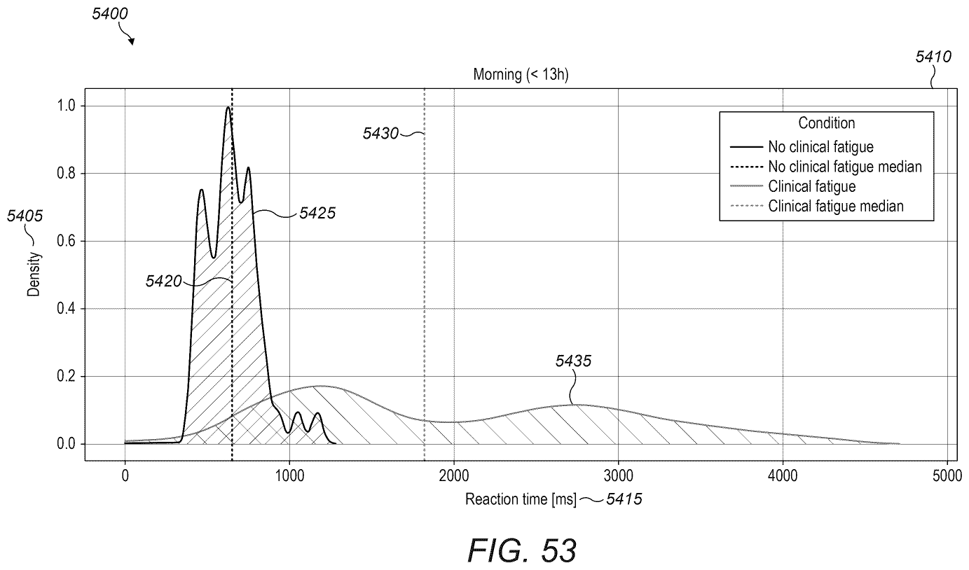

Turning to FIG. 53, shown is a graph 5400 of a plot 5410 of distribution reaction time during morning hours by condition where the x-axis is reaction time in milliseconds 5415 and the y-axis is density of the data 5405. The no clinical fatigue condition 5425 is shown under the black line and the clinical fatigue condition 5435 is shown under the gray line. Also shown is the no clinical fatigue median 5420 and the clinical fatigue median 5430. The density of the data 5405 is set such that the area under the curve of each condition is set to 1.

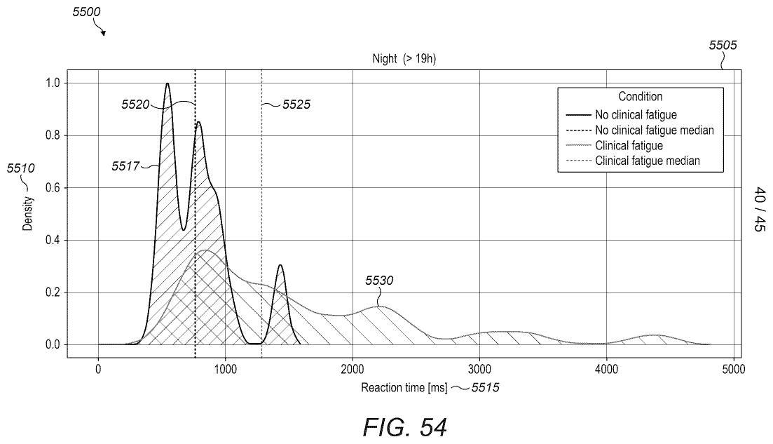

Turning to FIG. 54, shown is a graph 5500 of a plot 5505 of distribution reaction time during evening hours by condition where the x-axis 5515 is reaction time in milliseconds 5515 and the y-axis is density of the data 5510. The no clinical fatigue condition 5517 is shown under the black line and the clinical fatigue condition 5530 is shown under the gray line. Also shown is the no clinical fatigue median 5520 and the clinical fatigue median 5525. The density of the data 5510 is set such that the area under the curve of each condition is 1.

FIGS. 52, 53, 54 show the significant difference in reaction time between no clinical fatigue via CLD and clinical fatigue via CLD. Therefore, there is evidence that ground truth of self-reporting of fatigue for those without a clinical fatigue diagnosis is useful.

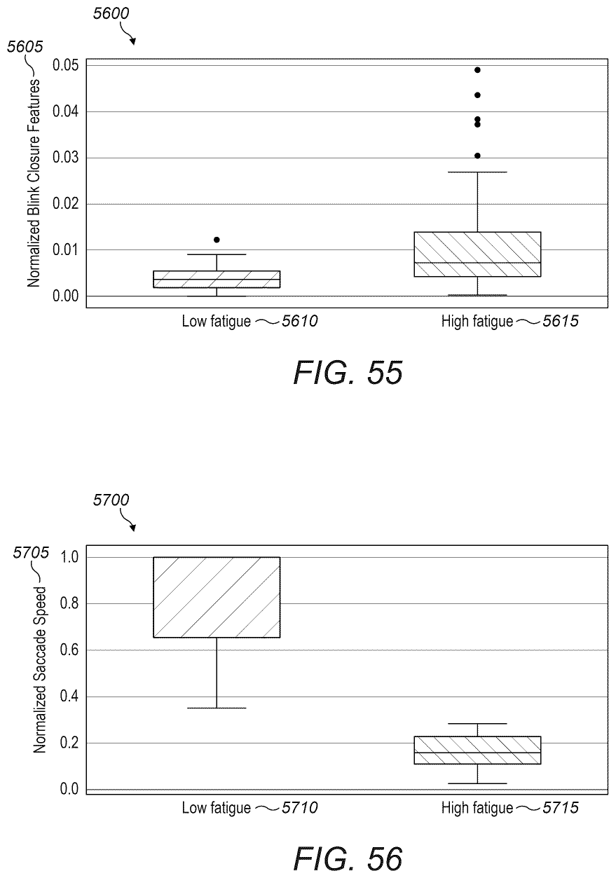

Turning to FIG. 55, shown is a boxplot 5600 of Normalized Blink Closure Features 5605 for low fatigue levels 5610 and high fatigue levels 5620.

Turning to FIG. 56, shown is a boxplot 5700 of Normalized Saccade 5705 for low fatigue levels 5720 and high fatigue levels 5730.

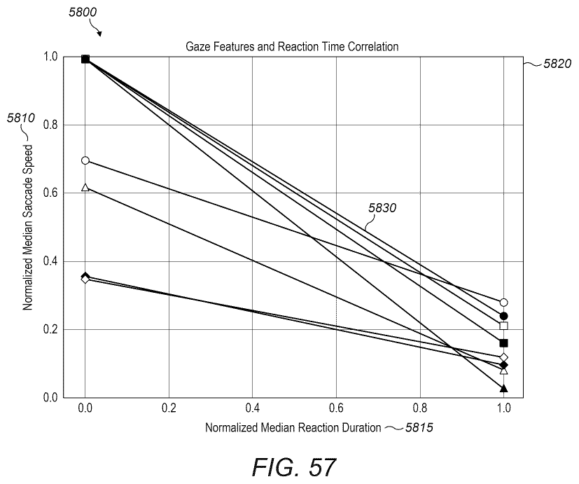

Turning to FIG. 57, shown is a graph 5800 showing a correlation plot 5820 with the x-axis as normalized median reaction duration 5815 and the y-axis as normalized median saccade speed 5810. The correlation lines 5830 show a significant negative correlation.

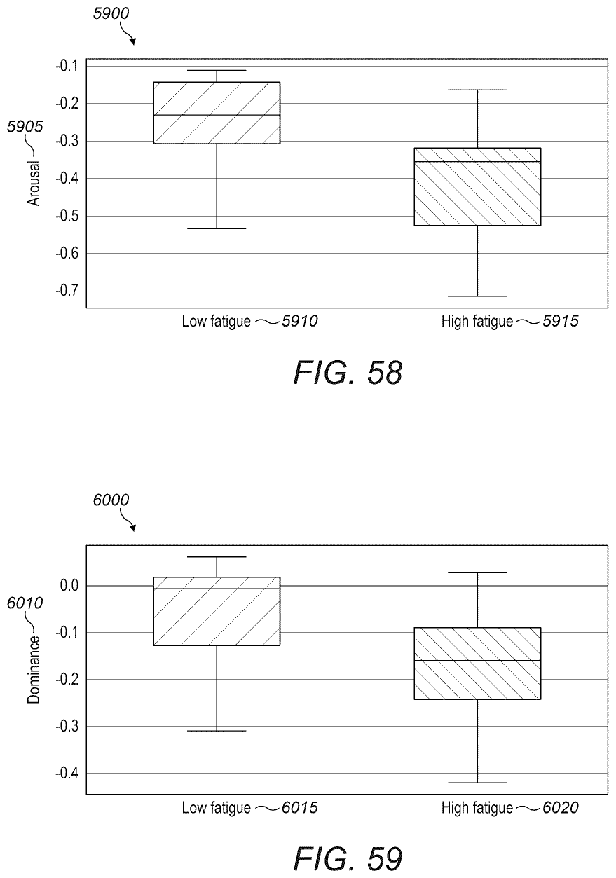

Turning to FIG. 58, shown is a boxplot 5900 of Arousal 5905 for low fatigue levels 5910 and high fatigue levels 5915.

Turning to FIG. 59, shown is a boxplot 6000 of Dominance 6010 for low fatigue levels 6015 and high fatigue levels 6020.

The evaluation results presented above indicate a strong correlation between the following fatigue markers and fatigue labels in this dataset: Blink closure; Saccade velocity; Voice arousal; and Voice dominance.

D. Simulator Drowsiness Validation

1. Drowsiness Detection model input features

Below is a list of features used by the model for drowsiness detection. These features include: AU 1; AU 2; AU 4; AU 5; AU 6; AU 10; AU 12; AU 14; AU 15; AU 23; AU 25; AU 45; Blink amplitude; Eye opening velocity; Eye closing velocity; Head pose pitch; Gaze yaw; Gaze pitch; Mouth aspect ratio (MAR); and Valence.

The statistics of the above features (mean, standard deviation, minimum, and maximum) are computed to form one part of the inputs into the drowsiness detection model.

The rest of the model inputs are formed from the statistics (mean, standard deviation, maximum, and minimum) of the difference between consecutive frames of the following features: AU 1; AU 2; AU 5; AU 6; AU 7; AU 9; AU 10; AU 12; AU 14; AU 15; AU 17; AU 23; AU 45; Head pose yaw; Head pose pitch; Head pose roll; Gaze yaw; Gaze pitch; Mouth aspect ratio (MAR); Valence; and Arousal.

Table 2 containing the full list of drowsiness detection model inputs are provided below:

TABLE 2

| Feature | Mean | Standard Dev. | Min. | Max. |
|---|---|---|---|---|
| AU 1 | ✓ | ✓ | ✓ | ✓ |
| AU 2 | ✓ | | ✓ | ✓ |
| AU 4 | | ✓ | | |
| AU 5 | | ✓ | | |
| AU 6 | | | ✓ | ✓ |
| AU 9 | ✓ | | | ✓ |
| AU 10 | | ✓ | | |
| AU 12 | ✓ | | | ✓ |
| AU 14 | ✓ | ✓ | | ✓ |
| AU 15 | | ✓ | ✓ | ✓ |
| AU 17 | | ✓ | ✓ | ✓ |
| AU 23 | | ✓ | | |
| AU 25 | ✓ | | | |
| AU 45 | | ✓ | ✓ | |
| AU 1 (difference) | | ✓ | | ✓ |
| AU 2 (difference) | ✓ | | | |
| AU 5 (difference) | ✓ | | ✓ | |
| AU 6 (difference) | | | ✓ | |
| AU 7 (difference) | ✓ | | | ✓ |
| AU 9 (difference) | | | ✓ | ✓ |
| AU 10 (difference) | | ✓ | ✓ | ✓ |
| AU 12 (difference) | | ✓ | ✓ | ✓ |
| AU 14 (difference) | ✓ | | | |
| AU 15 (difference) | | | | ✓ |
| AU 17 (difference) | ✓ | ✓ | ✓ | ✓ |
| AU 23 (difference) | ✓ | | | |
| AU 45 (difference) | ✓ | ✓ | | |
| Blink amplitude | ✓ | ✓ | | |
| Eye opening velocity | | ✓ | | |
| Eye closing velocity | ✓ | | | |
| Head pose pitch | ✓ | | ✓ | |
| Head pose yaw (difference) | | ✓ | | ✓ |
| Head pose pitch (difference) | ✓ | ✓ | ✓ | ✓ |
| Head pose roll (difference) | | | | ✓ |
| Gaze yaw | ✓ | ✓ | | |
| Gaze pitch | | ✓ | | ✓ |
| Gaze yaw (difference) | | ✓ | ✓ | |
| Gaze pitch (difference) | | ✓ | ✓ | |
| MAR | ✓ | | | ✓ |
| MAR (difference) | | ✓ | ✓ | ✓ |
| Valence | | | | ✓ |
| Valence (difference) | ✓ | | ✓ | |
| Arousal (difference) | ✓ | ✓ | ✓ | ✓ |

2. Study Analysis

A validation study was conducted in accordance with the European Union Regulation (EU) 2021/1341, whereby twenty drivers undertook an extended drive (lasting up to one hour) using the car-following paradigm in a driving simulator.

The Karolinska Sleepiness Scale (KSS) measures the subjective level of sleepiness at a particular time during the day. On this scale, subjects indicate a level which best reflects the psycho-physical state experienced in the last 10 minutes. The KSS is a measure of situational sleepiness.

Scoring of KSS is on a 9 point scale:
1=extremely alert
2=very alert
3=alert
4=rather alert
5=neither alert nor sleepy
6=some signs of sleepiness
7=sleepy, but no effort to keep awake
8=sleepy and some effort to keep awake
9=very sleepy, great effort to keep awake, fighting sleep

The ground truth for the dataset was self-reported KSS,, and the model aimed to detect instances of KSS values higher or equal to 8.

For the A-pillar camera position the drowsiness model achieved an accuracy of 69.2% of when drivers were reportedly drowsy (sensitivity). In addition, the model correctly identified 77.2% of instances when drivers were reportedly alert (specificity).

The results below in Table 3 contains the sensitivity and specificity results for the right A pillar camera, for 13 participants (7 were removed for either not reaching the drowsiness threshold according to the protocol or a technical issue with the recording). The protocol requires 10 valid participants.

TABLE 3

| | camera_IR_RAPL_task_drowsiness.mp4 | | | | | |
|---|---|---|---|---|---|---|
| Participant No. | TP | FP | TN | FN | Sensitivity | Specificity |
| P01 | 1 | 4 | 1 | | 100.0% | 20.0% |
| P02 | 1 | | 4 | | 100.0% | 100.0% |
| P04 | 1 | 2 | 6 | | 100.0% | 75.0% |
| P06 | 1 | | 18 | | 100.0% | 100.0% |
| P07 | | | 7 | 3 | 0.0% | 100.0% |
| P08 | | | 7 | 3 | 0.0% | 100.0% |
| P09 | | | 17 | 1 | 0.0% | 100.0% |
| P12 | 1 | 6 | 4 | | 100.0% | 40.0% |
| P13 | 1 | 4 | 3 | | 100.0% | 42.9% |
| P14 | 1 | 2 | 12 | | 100.0% | 85.7% |
| P16 | | | 9 | 2 | 0.0% | 100.0% |
| P18 | 1 | 9 | 6 | | 100.0% | 40.0% |
| P19 | 1 | | 13 | | 100.0% | 100.0% |
| Mean | | | | | 69.2% | 77.2% |
| Std Dev | | | | | 48.0% | 30.2% |

In Table 3, TP means True Positives, FP means False Positives, TN means Ture Negatives, False Negatives means.

3. In-car Drowsiness Validation (Zenzic)

This section describes the evaluation results of our drowsiness detection model on a real-world driving video dataset. The following results were obtained from a data set of 7 participants, collected in car, while driving for up to 4 hours with associated self-reported KSS ratings collected every 10 minutes.

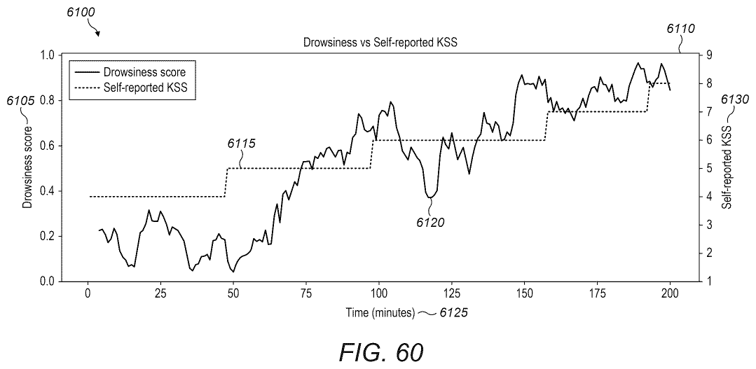

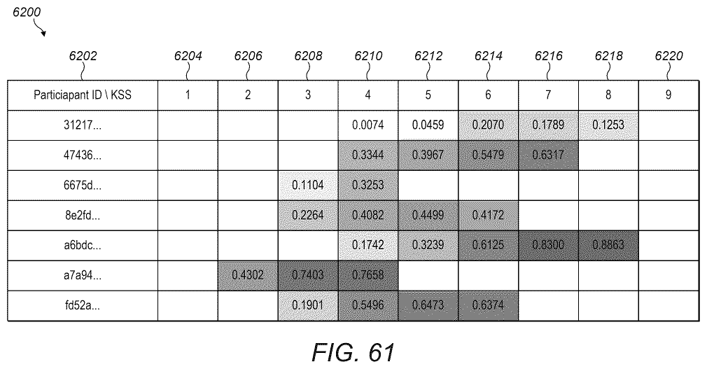

Turing to FIG. 60, shown is a temporal graph 6100 of a plot 6110 with the x-axis in minutes 6125, the left y-axis in drowsiness score 6105, and the right y-axis is self-reported KSS 6130. The dashed line 6115 shows the self-reported KSS and the sold line 6120 is the drowsiness score. FIG. 60 illustrates the progression of self-reported KSS scale and the drowsiness model score for one of the few subjects with the KSS score above 7. The strong correlation between the model scores and the self-reported scores illustrates the ability of the model to detect drowsiness in real-world driving conditions, despite the challenges in terms of face visibility, head movements, etc.

Turning to FIG. 61, shown is columnar chart 6200 showing anonymized participant IDs 6202 and 9 KSS scores 6204, 6206, 6208, 6210, 6212, 6214, 6216, 6218, 6220.

FIG. 61 shows the progression of the average drowsiness scores from the model in comparison with KSS ratings. Scores are computed from all valid frames i.e., frames in which face is clearly visible, from each model. Grayed out cells indicate there are no valid frames for this KSS value in the recordings.

Strong correlation between the model scores and the KSS ratings for most subjects shows that the model's predictions generalize reasonably well across different subjects.

Posing this as a binary classification problem to detect drowsy or non-drowsy states, we evaluated the model's accuracy in terms of F1 score. F1 is a metric that measures the model's accuracy performance by calculating the harmonic mean of the precision and recall of the model. F1 is calculated as follows:

$$F 1 = 2 \frac{precision * recall}{precision + recall}$$

F1 is a commonly used because it reliably measures the accuracy of the model regardless of the imbalanced nature of datasets. Higher is better.

For this analysis, we applied a threshold of 7 on the KSS scale for drowsy and non-drowsy categorization. This resulted in an average F1 Score of 0.47 for drowsiness detection.

The dataset had some label noise, resulting in this seemingly low score.

### ADDITIONAL DISCLOSURE

1. A method of estimating fatigue level in a subject, comprising: collecting subject video; using a face detection module to obtain face detection video data from the subject video; using a landmark detection module to obtain landmark detection video data from the face detection video data; using a gaze estimation module to obtain gaze estimation video data from the face detection video data; using a head pose module to obtain head pose video data from the face detection video data; using an action unit module to obtain action unit video data from the face detection video data; collecting subject audio; using an audio detection module to obtain audio detection data from the subject audio; and estimating fatigue in the subject based on the landmark detection video data, the gaze estimation video data, the head pose video data, and the audio detection data.

2. The method as in claim 1, wherein the estimating fatigue in the subject comprises measuring reaction time of the subject.

3. The method as in claim 2, wherein the measuring reaction time of the subject comprises analyzing time difference stimulus presentation and reaction of the subject.

4. The method as in claim 1, wherein the estimating fatigue in the subject comprises tracking a gaze of the subject.

5. The method as in claim 4, wherein the tracking the gaze of the subject. comprises analyzing saccade angular velocity of the subject and analyzing blink phase duration of the subject.

6. The method as in claim 1, wherein the estimating fatigue in the subject comprises measuring vocal characteristics of the subject.

7. The method as in claim 6, wherein the measuring vocal characteristics of the subject comprises analyzing saccade angular velocity of the subject, analyzing blink phase duration of the subject, analyzing loudness variability of the subject, and analyzing speech articulation rate of the subject.

8. The method as in claim 1, wherein the estimating fatigue in the subject comprises measuring facial muscle dynamics of the subject.

9. The method as in claim 1, wherein the estimating fatigue in the subject comprises measuring vocal descriptions provided by the subject.

10. The method as in claim 9, wherein the measuring vocal descriptions comprises computing statistical features for valence, arousal, and dominance.

11. The method as in claim 9, wherein the measuring vocal descriptions provided by the subject comprises analyzing facial muscle activations of the subject, analyzing facial expression intensity of the subject, analyzing blink phase duration of the subject, analyzing loudness variability of the subject, and analyzing speech articulation rate of the subject.

12. The method as in claim 1, wherein the estimating fatigue in the subject comprises deriving a confidence level for the estimating fatigue in the subject.

13. The method as in claim 1, further comprising: prior to the collecting subject video and the collecting subject audio, providing training to a depression model using mood diary tasks and read-aloud tasks; and wherein the estimating fatigue in the subject is also based on the depression model.

14. The method as in claim 1, wherein the action unit video data comprises analysis related to at least one of: AU 6 (Cheek Raiser); AU 10 (Upper Lip Raiser); AU 12 (Lip Corner Puller); AU 14 (Dimpler); AU 15 (Lip Corner Depressor); AU 17 (Chin Raiser); and AU 23 (Lip Tightener).

15. The method as in claim 1, wherein the audio detection data comprises analysis related to mean pause count, loudness, and pitch.

16. The method as in claim 1, wherein the collecting subject video and the collecting subject audio occurs while the subject is operating a vehicle.

17. The method as in claim 1, wherein the collecting subject video and the collecting subject audio occurs while the subject is providing input to an app.

### REFERENCES

[1] Jackson, Craig. “The Chalder fatigue scale (CFQ 11).” Occupational medicine 65.1 (2015): 86-86.

[2] Schleicher, Robert, et al. “Blinks and saccades as indicators of fatigue in sleepiness warnings: looking tired?.” Ergonomics 51.7 (2008):982-1010.

[3] Schmidt, D., et al. “Saccadic velocity characteristics-Intrinsic variability and fatigue.” Aviation, space, and environmental medicine 50.4 (1979): 393-395.

[4] Stern, John A., Donna Boyer, and David Schroeder. “Blink rate: a possible measure of fatigue.” Human factors 36.2 (1994): 285-297.

[5] Vural, Esra, et al. “Drowsy driver detection through facial movement analysis.” Human-Computer Interaction: IEEE International Workshop, HCI 2007 Rio de Janeiro, Brazil, Oct. 20, 2007, Proceedings 4. Springer Berlin Heidelberg, 2007.

[6] Greeley, Harold P., et al. “Fatigue estimation using voice analysis.” Behavior research methods 39 (2007): 610-619.

[7] Russell, James A. “A circumplex model of affect.” Journal of personality and social psychology 39.6 (1980): 1161.

[8] Espinosa, J., Domenech, B., Vázquez, C., Pérez, J., & Mas, D. (2018). Blinking characterization from high speed video records. Application to biometric authentication. PloS one, 13(5), e0196125.

[9] Huang, Z., Tang, W., Tian, Q., Huang, T., & Li, J. (2024). Air Traffic Controller Fatigue Detection Based on Facial and Vocal Features Using Long Short-Term Memory. IEEE Access.

[10] Hu, Y., Liu, Z., Hou, A., Wu, C., Wei, W., Wang, Y., & Liu, M. (2022). On fatigue detection for air traffic controllers based on fuzzy fusion of multiple features. Computational and mathematical methods in medicine, 2022.

[11] Craye, C., Rashwan, A., Kamel, M. S., & Karray, F. (2016). A multi-modal driver fatigue and distraction assessment system. International Journal of Intelligent Transportation Systems Research, 14, 173-194.

[12] Naeeri, S., Kang, Z., Mandal, S., & Kim, K. (2021). Multimodal analysis of eye movements and fatigue in a simulated glass cockpit environment. Aerospace, 8(10), 283.

[13] Brunet et al., Validation of sleep-2-Peak: A smartphone application that can detect fatigue-related changes in reaction times during sleep deprivation, Behav Res (2017) 49:1460-1469.

[14] Big Five Inventory (John, O. P., & Srivastava, S. (1999). The Big-Five trait taxonomy: History, measurement, and theoretical perspectives. In L. A. Pervin & O. P. John (Eds.), Handbook of personality: Theory and research (Vol. 2, pp. 102-138). New York: Guilford Press).

### CONCLUSION

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The term “coupled” as used herein is defined as connected, although not necessarily directly and not necessarily mechanically. A device or structure that is “configured” in a certain way is configured in at least that way but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A method of estimating fatigue level in a subject, comprising:
collecting subject video;
using a face detection module to obtain face detection video data from the subject video;
using a landmark detection module to obtain landmark detection video data from the face detection video data;
using a gaze estimation module to obtain gaze estimation video data from the face detection video data;
using a head pose module to obtain head pose video data from the face detection video data;
using an action unit module to obtain action unit video data from the face detection video data;
collecting subject audio;
using an audio detection module to obtain audio detection data from the subject audio; and
estimating fatigue in the subject based on the landmark detection video data, the gaze estimation video data, the head pose video data, and the audio detection data.

2. The method as in claim 1, wherein the estimating fatigue in the subject comprises measuring reaction time of the subject.

3. The method as in claim 2, wherein the measuring reaction time of the subject comprises analyzing time difference stimulus presentation and reaction of the subject.

4. The method as in claim 1, wherein the estimating fatigue in the subject comprises tracking a gaze of the subject.

5. The method as in claim 4, wherein the tracking the gaze of the subject. comprises analyzing saccade angular velocity of the subject and analyzing blink phase duration of the subject.

6. The method as in claim 1, wherein the estimating fatigue in the subject comprises measuring vocal characteristics of the subject.

7. The method as in claim 6, wherein the measuring vocal characteristics of the subject comprises analyzing saccade angular velocity of the subject, analyzing blink phase duration of the subject, analyzing loudness variability of the subject, and analyzing speech articulation rate of the subject.

8. The method as in claim 1, wherein the estimating fatigue in the subject comprises measuring facial muscle dynamics of the subject.

9. The method as in claim 1, wherein the estimating fatigue in the subject comprises measuring vocal descriptions provided by the subject.

10. The method as in claim 9, wherein the measuring vocal descriptions comprises computing statistical features for valence, arousal, and dominance.

11. The method as in claim 9, wherein the measuring vocal descriptions provided by the subject comprises analyzing facial muscle activations of the subject, analyzing facial expression intensity of the subject, analyzing blink phase duration of the subject, analyzing loudness variability of the subject, and analyzing speech articulation rate of the subject.

12. The method as in claim 1, wherein the estimating fatigue in the subject comprises deriving a confidence level for the estimating fatigue in the subject.

13. The method as in claim 1, further comprising:
prior to the collecting subject video and the collecting subject audio, providing training to a depression model using mood diary tasks and read-aloud tasks; and
wherein the estimating fatigue in the subject is also based on the depression model.

14. The method as in claim 1, wherein the action unit video data comprises analysis related to at least one of: AU 6 (Cheek Raiser); AU 10 (Upper Lip Raiser); AU 12 (Lip Corner Puller); AU 14 (Dimpler); AU 15 (Lip Corner Depressor); AU 17 (Chin Raiser); and
AU 23 (Lip Tightener).

15. The method as in claim 1, wherein the audio detection data comprises analysis related to mean pause count, loudness, and pitch.

16. The method as in claim 1, wherein the collecting subject video and the collecting subject audio occurs while the subject is operating a vehicle.

17. The method as in claim 1, wherein the collecting subject video and the collecting subject audio occurs while the subject is providing input to an app.
